SEQUENCE LISTING (4 sequences)

SEQ ID NO: 1 - protein, 571 aa, Unknown
  Adenovirus serotype 5 penton base sequence
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 2 - protein, 207 aa, Homo sapiens
ELLPPQLKEMKSQESAAGSKLVLRCETSSEYSSLRFKWFKNGNELNRKNKPQNIKIQKKPGKSELRINKASLADSGEYMCKVISKLGNDSASANIAIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELY

SEQ ID NO: 3 - protein, 796 aa, Artificial Sequence
  Synthetic Construct
ELLPPQLKEMKSQESAAGSKLVLRCETSSEYSSLRFKWFKNGNELNRKNKPQNIKIQKKPGKSELRINKASLADSGEYMCKVISKLGNDSASANIAIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYGGSGGSRSMRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTFKKKKKKKKKK

SEQ ID NO: 4 - protein, 10 aa, Artificial Sequence
  Synthetic Construct
KKKKKKKKKK

(19) United States
(12) Patent Application Publication
(10) Pub. No.: US 2019/0240344 A1
(43) Pub. Date: Aug. 8, 2019

(54) FUNCTIONAL RNA AND SMALL-MOLECULE DRUG THERAPEUTIC COMPLEXES AND NANOPARTICLE DELIVERY VEHICLES

(71) Applicant: EOS BIOSCIENCES, INC., Century City, CA (US)

(72) Inventor: Omar K. HAFFAR, Century City, CA (US)

(21) Appl. No.: 16/338,909

(22) PCT Filed: Oct. 3, 2017
(86) PCT No.: PCT/US17/54884

§ 371 Date: Apr. 2, 2019

Related U.S. Application Data
(60) Provisional application No. 62/403,595, filed on Oct. 3, 2016.

(51) Int. Cl. A61K 47/64; A61K 47/42; A61K 47/54; A61K 47/55; A61K 31/713

(52) U.S. Cl. CPC A61K 47/6455; A61K 47/42; A61K 38/17; A61K 47/55; A61K 31/713; A61K 47/549

(57) ABSTRACT

Disclosed herein are therapeutic complexes comprising a small-molecule drug complexed with a functional RNA. Further disclosed herein are compositions comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment, along with methods of making and using such nanoparticles. Further described are methods of treating a subject with a cancer, comprising administering to the subject an effective amount of a composition comprising nanoparticles, the nanoparticles comprising a carrier polypeptide and a functional RNA molecule complexed with a small-molecule chemotherapeutic drug, wherein the carrier polypeptide comprises a cell-targeting segment, a cell-penetrating segment, and an oligonucleotide-binding segment. Also described are pharmaceutical compositions, articles of manufacture, and kits comprising the described nanoparticles.

### CROSS-REFERENCE TO RELATED APPLICATION

This application claims priority benefit to U.S. Provisional Application No. 62/403,595, filed on Oct. 3, 2016, entitled “DRUG-DELIVERY NANOPARTICLES WITH RNA AND SMALL-MOLECULE CARGOS,” which is incorporated herein by reference for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name 761542000840SEQLIST.txt, date recorded: Oct. 3, 2017, size: 14 KB).

### FIELD OF THE INVENTION

The present invention relates to the methods and nanoparticle compositions for the treatment of cancer. The present invention further relates to nucleic acid-drug complexes.

### BACKGROUND

Current strategies for targeting therapy to tumors include antibody-targeted chemotherapy agents (i.e., immunoconjugates), targeted toxins, signal-blocking antibodies, and antibody-targeted liposomes (i.e., immunoliposomes). Trastuzumab, for example, is a monoclonal antibody that interferes with HER2/neu signaling, and is commonly used for the treatment of HER2+ breast cancer. However, trastuzumab-resistant cancers can also arise after the start of treatment, limiting the efficacy of the therapeutic.

Small-molecule chemotherapeutics, such as doxorubicin, are also commonly used to treat certain cancers. But doxorubicin also poses significant risk of cardiomyopathy and cancer resistance. Delivery of small-molecule drugs, such as doxorubicin, through the use of liposomes (such as LipoDox) has improved the effectiveness of administering the drug for certain cancers. Still, the toxicity of many anticancer agents presents a pressing need for effective low-dose therapeutics.

The disclosures of all publications, patents, and patent applications referred to herein are hereby incorporated herein by reference in their entireties.

### SUMMARY OF THE INVENTION

In some aspect, there is provided a composition comprising a functional RNA molecule complexed with a small-molecule drug, wherein the functional RNA molecule modulates expression of a target protein.

In another aspect, there is provided a functional RNA molecule comprising at least one complementary region intercalated with a small-molecule drug. In some embodiments, the functional RNA molecule modulates expression of a target protein.

In some embodiments of the above compositions, the composition comprises a liposome containing the functional RNA molecule and the small molecule drug. In some embodiments, the liposome comprises a cell targeting segment.

In another aspect, there is provided a composition comprising nanoparticles comprising a carrier polypeptide and a functional RNA molecule complexed with a small-molecule drug, wherein the carrier polypeptide comprises a cell-penetrating segment and an oligonucleotide-binding segment. In some embodiments, the carrier polypeptide further comprises a cell-targeting segment.

In some embodiments, the small-molecule drug is intercalated into the RNA molecule. In some embodiments, at least a portion of the RNA molecule is double stranded. In some embodiments, the RNA molecule is single stranded and comprises at least one self-complementary region. In some embodiments, the RNA molecule is siRNA, shRNA, miRNA, circularRNA (circRNA), rRNA, Piwi-interacting RNA (piRNA), toxic small RNA (tsRNA), or a ribozyme. In some embodiments, the RNA molecule is an antisense RNA molecule. In some embodiments, the RNA molecule has at least one triphosphate 5′-end. In some embodiments, the RNA molecule is about 10 nucleotides to about 100 nucleotides in length. In some embodiments, the molar ratio of the RNA molecule to the small-molecule drug in the nanoparticle composition is about 1:1 to about 1:60. In some embodiments, the molar ratio of the functional RNA molecule to the small-molecule drug in the composition is about 1:5 to about 1:60.

In some embodiments, the functional RNA molecule decreases expression of an immune checkpoint protein.

In some embodiments, the small-molecule drug is a chemotherapeutic agent. In some embodiments, the small-molecule drug is an anthracycline. In some embodiments, the small-molecule drug is doxorubicin. In some embodiments, the small-molecule drug is an alkylating agent or an alkylating-like agent. In some embodiments, the small-molecule drug is of Carboplatin, Carmustine, Cisplatin, Cyclophosphamide, Melphalan, Procarbazine, or Thiotepa.

In some embodiments, the molar ratio of carrier polypeptide to RNA molecule in the composition is about 3:1 to about 8:1. In some embodiments, the molar ratio of carrier polypeptide to RNA molecule in the composition is about 4:1 to about 5:1. In some embodiments, the molar ratio of carrier polypeptide to RNA molecule in the composition is about 4:1.

In some embodiments, the cell-targeting segment binds a mammalian cell. In some embodiments, the cell-targeting segment binds a diseased cell. In some embodiments, the cell-targeting segment binds a cancer cell. In some embodiments, the cancer cell is a HER3+ cancer cell or a c-MET+ cancer cell. In some embodiments, the cancer cell is a head and neck cancer cell, a pancreatic cancer cell, a breast cancer cell, a glial cancer cell, an ovarian cancer cell, a cervical cancer cell, a gastric cancer cell, a skin cancer cell, a colon cancer cell, a rectal cancer cell, a lung cancer cell, a kidney cancer cell, a prostate cancer cell, or a thyroid cancer cell.

In some embodiments, the cell-targeting segment binds a target molecule on the surface of a cell. In some embodiments, the cell-targeting segment binds a receptor on the surface of a cell. In some embodiments, the cell-targeting segment binds HER3 or c-MET.

In some embodiments, the cell-targeting segment comprises a ligand that specifically binds to a receptor expressed on the surface of a cell. In some embodiments, the cell-targeting segment comprises a heregulin sequence or a variant thereof; or an Internalin B sequence or a variant thereof. In some embodiments, the cell-targeting segment comprises a receptor binding domain of heregulin-α.

In some embodiments, the cell-penetrating segment comprises a penton base polypeptide or a variant thereof.

In some embodiments, the oligonucleotide-binding segment is positively charged. In some embodiments, the oligonucleotide-binding segment comprises polylysine. In some embodiments, the oligonucleotide-binding segment comprises decalysine.

In some embodiments, the carrier polypeptide is HerPBK10.

In some embodiments, the average size of the nanoparticles in the composition is about 100 nm or less.

In some embodiments, the composition is sterile. In some embodiments, the composition is a liquid composition. In some embodiments, the composition is a dry composition. In some embodiments, the composition is lyophilized.

In another aspect there is provided a pharmaceutical composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment, further comprising a pharmaceutically acceptable excipient.

In another aspect there is provided an article of manufacture comprising a composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment in a vial. In some embodiments, the vial is sealed.

In another aspect, there is provided a kit comprising a composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment, and an instruction for use.

In another aspect, there is provided a method of treating a cancer in a subject comprising administering an effective amount of the composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment to the subject. In some embodiments, the cancer is a HER3+ cancer or a c-MET+ cancer. In some embodiments, the cancer is a head and neck cancer, a pancreatic cancer, a breast cancer, an ovarian cancer, a glial cancer, a cervical cancer, a gastric cancer, a skin cancer, a colon cancer, a rectal cancer, a lung cancer, a kidney cancer, a prostate cancer cell, or a thyroid cancer.

In another aspect, there is provided a method of simultaneously modulating expression of a target protein and inhibiting growth of a cell, comprising administering any of the above-described compositions to the cell.

In another aspect, there is provided a method of simultaneously stimulating an immune response and killing a cancer cell in a subject with cancer, comprising administering an effective amount of the above-described composition to the subject. In some embodiments, the functional RNA molecule decreases expression of an immune checkpoint protein.

In another aspect, there is provided a method of making a composition, comprising combining a small-molecule drug with a functional RNA molecule, wherein the small-molecule drug intercalates into the functional RNA molecule. In some embodiments, there is provided a method of making a nanoparticle composition comprising combining a carrier polypeptide, a functional RNA molecule, and a small-molecule drug, wherein the carrier polypeptide comprises a cell-penetrating segment and an oligonucleotide-binding segment. In some embodiments, the method comprises combining the RNA molecule with the small-molecule drug to complex the drug to the RNA molecule; and combining the carrier polypeptide with the RNA molecule complexed with the small-molecule drug. In some embodiments, the method comprises removing unbound small-molecule drug. In some embodiments, the small-molecule drug intercalates the RNA molecule. In some embodiments, the method further comprises sterile filtering the nanoparticle composition. In some embodiments, the method further comprises lyophilizing the nanoparticle composition.

### BRIEF DESCRIPTION OF THE DRAWING

FIG. 1 illustrates a schematic of the carrier polypeptide comprising a cell-targeting domain, a cell-penetrating domain, and an oligonucleotide-binding domain. When carrier polypeptides are combined with the functional RNA molecules bound to a small-molecule drug, nanoparticles are formed.

FIG. 2 shows a 1% agarose gel loaded with dox:siRNA1 complex and dox:siRNA2 complex samples prior to filtration (lanes 2 and 3), and the retentate (lanes 5 and 6) and filtrate (lanes 7 and 8) after filtration using a 10K MWCO filter. The pre-filtered complexes and the retentates include the siRNA, whereas the filtrate does not.

FIG. 3 shows absorbance spectra of the retentate and filtrate from the dox:siRNA1 complex (top) and the dox:siRNA2 complex (bottom) after filtration on a 10K MWCO filter. The retentate for both complexes has a maximum peak at approximately 480 nm, indicating the doxorubicin was present in the retentate. The filtrate did not have a significant peak at 480 nm, indicating little doxorubicin in the filtrate.

FIG. 4 shows absorbance spectra of the retentate and filtrate from the dox:siScrm1 complex, the dox:siRNA1 complex, the dox:siRNA2 complex, and the dox:DNA oligo complex after filtration on a 10K MWCO filter. The retentate of all four complexes has a maximum peak at approximately 480 nm, indicating the doxorubicin was present in the retentate. The filtrate did not have a significant peak at 480 nm, indicating little doxorubicin in the filtrate.

FIGS. 5A-C show cell viability of JIMT1 cells after transfection with three different doses of siScrm1, siRNA1, siRNA2, dox:siScrm1 complex, dox:siRNA1 complex, dox:siRNA2 complex, dox:DNA oligo complex, or doxorubicin alone after 24 hours (FIG. 5A), 48 hours (FIG. 5B), or 72 hours (FIG. 5C).

FIG. 6A shows relative mRNA knockdown of the siRNA1 target mRNA (measured by qPCR) 24 hours after transfecting JIMT1 (trastuzumb-resistant human breast cancer) cells with three different concentrations of siScrm1, siRNA1, siRNA2, dox:siScrm1 complex, dox:siRNA1 complex, dox:siRNA2 complex, dox:DNA oligo complex, or doxorubicin alone.

FIG. 6B shows relative mRNA knockdown of the siRNA2 target mRNA (measured by qPCR) 24 hours after transfecting JIMT1 cells with three different concentrations of siScrm1, siRNA1, siRNA2, dox:siScrm1 complex, dox:siRNA1 complex, dox:siRNA2 complex, dox:DNA oligo complex, or doxorubicin alone.

FIG. 7 shows absorbance spectra of the retentate and filtrate from the dox:siScrm2 complex and the dox:siRNA3 complex after filtration on a 10K MWCO filter. The retentate for both complexes has a maximum peak at approximately 480 nm, indicating the doxorubicin was present in the retentate. The filtrate did not have a significant peak at 480 nm, indicating little doxorubicin in the filtrate.

FIG. 8 show cell viability of 4T1-Fluc-Neo/eGFP-Puro cells after transfection with three different doses of siScrm2, siRNA3, dox:siScrm2 complex, dox:siRNA3 complex, or doxorubicin alone after 24 hours. 4T1-Fluc-Neo/eGFP-Puro cells are mouse mammary tumor line cells that stably express Fluc and eGFP. The 4T1 cell line is considered a triple negative mammary cancer cell line.

### DETAILED DESCRIPTION OF THE EMBODIMENTS

Provided herein are therapeutic complexes that include a functional RNA molecule (such as a double-stranded functional RNA molecule siRNA molecule) complexed to a small-molecule drug (such as a chemotherapeutic agent). In some embodiments, the small-molecule drug intercalates into the functional RNA molecule. The therapeutic complex can be delivered to a cell as a consolidated single delivery package, such as through the use of a liposome or nanoparticle delivery vehicle, which may be targeted to the cell. For example, in some aspects, the therapeutic complex is included in a liposome, which can deliver the complex to a cell (i.e., through lipofection). In certain aspects, there is provided a composition comprising nanoparticles comprising a carrier polypeptide and a functional RNA molecule complexed with a small-molecule drug. The carrier polypeptide includes a cell-penetrating segment and an oligonucleotide-binding segment, and can spontaneously assemble into nanoparticles when combined with the therapeutic complex. In some aspects, an effective amount of the nanoparticle composition or the therapeutic complex is administered to a subject with cancer to treat the cancer.

The simultaneous delivery of both the functional RNA molecule and the small-molecule drug to the cell (such as a cancer cell) allows for effective disease treatment while limiting undesirable side effects, such as a broad systemic immune response. Co-delivery of the small molecule drugs and the functional RNA molecules can act synergistically to effect a slowing of tumor growth or even tumor regression. Previous systems for the delivery of siRNA and doxorubicin, such as those described in Liu et al., Co-delivery of doxorubicin and siRNA by a simplified platform with oligodeoxynucleotides as a drug carrier, Colloids and Surfaces B: Biointerfaces, vol. 126, pp. 531-540 (2015), relied on intercalating doxorubicin into specifically designed DNA oligonucleotide containing CGA repeats (i.e., CGA-DNA oligonucleotides), and mixing the dox:DNA complex with PEI, CMCS-PEG-NGR, and siRNA to form dual-cargo particles (that is dox:DNA cargo and functional RNA cargo). As further detailed herein, it has been found that small-molecule drugs, such as doxorubicin, can intercalate functional RNA molecules, and that the complex retains both the functional properties of the functional RNA molecule and the small-molecule drug. Further, the small-molecule drug complexed to the functional RNA molecule results in increased potency of the small-molecule drug compared to the small-molecule drug administered alone. This surprising finding indicates that the small-molecule drug can bind nucleic acid molecules other than carefully designed CGA-DNA oligonucleotides. As this finding allows for direct binding of the small-molecule drug to RNA molecules, the RNA molecules can be designed to be functional, such as to modulate (i.e., increase or decrease) protein expression and/or have a biological effect (such as an anti-cancer effect). Further, the finding that the small-molecule drug can directly bind the functional RNA molecule allows for the simplified delivery of a single complex rather than a mixture of a dox:DNA complex and an siRNA.

In some embodiments, the complex is delivered to a cell using a carrier polypeptide, which can assemble into a nanoparticle. For example, provided herein are nanoparticle compositions comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment. The carrier polypeptide of the nanoparticles can protect, transport, and target the functional RNA molecule and the small-molecule drug to a targeted cell, such as a cancer cell. The carrier polypeptide includes a cell-penetrating segment, which allows for delivery of the functional RNA molecule and small-molecule drug to the interior of the cell. The nanoparticle can therefore ensure efficient, targeted delivery of the therapeutic complex to lower the effective dosage administered to a subject. Further, the carrier polypeptide protects the functional RNA molecule from extracellular nucleases or other factors that may degrade the functional RNA molecule.

In some embodiments, there is provided a method of simultaneously modulating expression of a target protein and inhibiting growth of a cell, comprising administering to the cell an effective amount of a composition comprising a functional RNA molecule complexed with a small-molecule drug (such as a chemotherapeutic drug). In some embodiments, the functional RNA molecule is a double stranded functional RNA molecule (such as double stranded siRNA). In some embodiments, the small-molecule drug is intercalated into the functional RNA molecule.

In some embodiments, there is provided a method of simultaneously modulating expression of a target protein and inhibiting growth of a cell, comprising administering to the cell an effective amount of a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule (such as double stranded siRNA). In some embodiments, the small-molecule drug is intercalated into the functional RNA molecule.

In some embodiments, there is provided a method of killing a cell, comprising transfecting the cell with a complex comprising a functional RNA molecule and a small-molecule drug (such as a chemotherapeutic drug). In some embodiments, the functional RNA molecule is a double stranded functional RNA molecule (such as double stranded siRNA). In some embodiments, the small-molecule drug is intercalated into the functional RNA molecule.

In some embodiments, there is provided a method of killing a cell, comprising administering to the cell a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule (such as double stranded siRNA). In some embodiments, the small-molecule drug is intercalated into the functional RNA molecule.

In some embodiments, there is provided a method of inducing apoptosis of a cell, comprising transfecting the cell with a complex comprising a functional RNA molecule and a small-molecule drug. In some embodiments, the functional RNA molecule is a double stranded functional RNA molecule. In some embodiments, the small-molecule drug is a chemotherapeutic agent. In some embodiments, the small-molecule drug is intercalated into the functional RNA molecule.

In some embodiments, there is provided a method of inducing apoptosis of a cell, comprising administering to the cell a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule. In some embodiments, the small-molecule drug is a chemotherapeutic agent. In some embodiments, the small-molecule drug is intercalated into the functional RNA molecule.

In some embodiments, there is provided a method of inducing necrosis of a cell, comprising transfecting the cell with a complex comprising a functional RNA molecule and a small-molecule drug. In some embodiments, the functional RNA molecule is a double stranded functional RNA molecule. In some embodiments, the small-molecule drug is a chemotherapeutic agent. In some embodiments, the small-molecule drug is intercalated into the functional RNA molecule.

In some embodiments, there is provided a method of inducing necrosis of a cell, comprising administering to the cell a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule. In some embodiments, the small-molecule drug is a chemotherapeutic agent. In some embodiments, the small-molecule drug is intercalated into the functional RNA molecule.

In some embodiments, there is provided a method of sensitizing a cancer cell to a chemotherapeutic drug, comprising administering to the cancer cell a composition comprising nanoparticles comprising a carrier polypeptide and a functional RNA molecule complexed with a chemotherapeutic drug, wherein the functional RNA molecule increases sensitivity of the cancer cell to the chemotherapeutic drug. In some embodiments, the functional RNA molecule is a double stranded functional RNA molecule (such as double stranded siRNA). In some embodiments, the functional RNA molecule is a siRNA molecule that decreases expression of a protein associated with drug efflux, chemotherapeutic drug resistance, or chemotherapeutic drug sensitivity. In some embodiments, the chemotherapeutic drug is intercalated into the functional RNA molecule.

In some embodiments, there is provided a method of sensitizing a cancer cell to a chemotherapeutic drug, comprising transfecting the cell with a complex comprising a functional RNA molecule and a chemotherapeutic drug, wherein the functional RNA molecule increases sensitivity of the cancer cell to the chemotherapeutic drug. In some embodiments, the functional RNA molecule is a double stranded functional RNA molecule (such as double stranded siRNA). In some embodiments, the functional RNA molecule is a siRNA molecule that decreases expression of a protein associated with drug efflux, chemotherapeutic drug resistance, or chemotherapeutic drug sensitivity. In some embodiments, the chemotherapeutic drug is intercalated into the functional RNA molecule

In some embodiments, there is provided a method of simultaneously modulating an immune response and killing a cancer cell, comprising administering to the cell an effective amount of a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule (such as double stranded siRNA). For example, in some embodiments, the functional RNA molecule is a siRNA molecule that decreases expression of an immune checkpoint protein. In some embodiments, the small-molecule drug is intercalated into the functional RNA molecule.

In some embodiments, there is provided a method of simultaneously modulating an immune response and killing a cancer cell, comprising transfecting the cell with a complex comprising a functional RNA molecule and a small-molecule drug (such as a chemotherapeutic drug). In some embodiments, the functional RNA molecule is a double stranded functional RNA molecule (such as double stranded siRNA). For example, in some embodiments, the functional RNA molecule is a siRNA molecule that decreases expression of an immune checkpoint protein. In some embodiments, the small-molecule drug is intercalated into the functional RNA molecule.

In some embodiments, there is provided a method of simultaneously modulating an immune response and killing a cancer cell in a subject with cancer, comprising administering to the subject an effective amount of a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule (such as double stranded siRNA). For example, in some embodiments, the functional RNA molecule is a siRNA molecule that decreases expression of an immune checkpoint protein. In some embodiments, the small-molecule drug is intercalated into the functional RNA molecule.

In some embodiments, there is provided a method of simultaneously modulating an immune response and killing a cancer cell in a subject with cancer, comprising administering to the subject an effective amount of a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule. For example, in some embodiments, the functional RNA molecule is a siRNA molecule that decreases expression of an immune checkpoint protein. In some embodiments, the small-molecule drug is a chemotherapeutic agent. In some embodiments, the small-molecule drug is intercalated into the functional RNA molecule.

In some embodiments, there is provided a method of treating cancer in a subject, comprising administering to the subject an effective amount of a complex comprising a functional RNA molecule and a small-molecule drug. In some embodiments, the functional RNA molecule is a double stranded functional RNA molecule. In some embodiments, the small-molecule drug is a chemotherapeutic agent. In some embodiments, the small-molecule drug is intercalated into the functional RNA molecule. In some embodiments, the complex is transported using a carrier, such as a liposome, a nanoparticle, or a carrier polypeptide.

In some embodiments, there is provided a method of treating cancer in a subject, comprising administering to the subject an effective amount of a composition comprising nanoparticles comprising a carrier polypeptide and a functional RNA molecule complexed with a small-molecule drug (such as a chemotherapeutic drug). In some embodiments, the functional RNA molecule is a double stranded functional RNA molecule. In some embodiments, the small-molecule drug is a chemotherapeutic agent. In some embodiments, the small-molecule drug is intercalated into the functional RNA molecule.

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”. Further, reference to “about X-Y” is equivalent to “about X to about Y,” and “about X-Y or Y-Z” is equivalent to “about X to about Y, or about Y to about Z.” Additionally, reference to “about X, Y, or Z or less” is equivalent to “about X or less, about Y or less, or about Z or less,” and reference to “about X, Y, or Z or more” is equivalent to “about X or more, about Y, or more, or about Z or more.”

The term “effective” is used herein, unless otherwise indicated, to describe an amount of a compound or component which, when used within the context of its use, produces or effects an intended result, whether that result relates to the treatment of an infection or disease state or as otherwise described herein.

The term “pharmaceutically acceptable” as used herein means that the compound or composition is suitable for administration to a subject, including a human subject, to achieve the treatments described herein, without unduly deleterious side effects in light of the severity of the disease and necessity of the treatment.

The term “subject” or “patient” is used synonymously herein to describe a mammal. Examples of a subject include a human or animal (including, but not limited to, dog, cat, rodent (such as mouse, rat, or hamster), horse, sheep, cow, pig, goat, donkey, rabbit, or primates (such as monkey, chimpanzee, orangutan, baboon, or macaque)).

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom, delay in progression of the disease, delay in recurrence of the disease, or inhibition of the disease.

A cell that exhibits upregulated expression for a particular protein (e.g., HER3+ or c-MET+) is said to be upregulated when the cell presents more of that protein relative to a cell that is not upregulated for that protein.

It is understood that aspects and variations of the invention described herein include “consisting” and/or “consisting essentially of” aspects and variations.

It is to be understood that one, some or all of the properties of the various embodiments described herein may be combined to form other embodiments of the present invention.

The section headings used herein are for organizational purposes only and are not to be construed as limiting the subject matter described.

### Functional RNA and Small-Molecule Drug Complexes

The therapeutic complex includes a functional RNA molecule complexed with a small-molecule drug. The small-molecule drug can complex with the functional RNA molecule, for example, by electrostatic interactions or by intercalating in the functional RNA molecule.

The functional RNA molecule can provide a biological function, such as causing inhibition of protein expression (for example, through an RNAi pathway), an increase in protein expression (for example, through the use of mRNA as the functional RNA molecule), or altered expression of one or more cytokines (such as a type I interferon (e.g., IFN-α, INF-β), IL-6, or IL-8)). In some embodiments, the functional RNA molecule is an anti-HER2 siRNA. In some embodiments, the functional RNA molecule modulates expression of an immune system checkpoint protein (e.g., programmed cell death protein ligand 1 (PD-L1), or programmed cell death protein 1 (PD-1), or cytotoxic T-lymphocyte-associated protein 4 (CTLA-4)) expressed by a tumor cell. In some embodiments, the functional RNA molecule is a siRNA molecule that decreases expression of an immune system checkpoint protein. In some embodiments, the functional RNA molecule modulates expression of a protein associated with drug efflux or drug resistance (such as a monocarboxylate transporter (MCT), a multiple drug resistance protein (MDR), a P-glycoprotein, a multidrug resistance-associated protein (MRP), a peptide transporter (PEPT), or a Na+ phosphate transporter (NPT)). In some embodiments, the functional RNA molecule is an siRNA molecule that decreases expression of a protein associated with drug efflux or drug resistance (such as a monocarboxylate transporter (MCT), a multiple drug resistance protein (MDR), a P-glycoprotein, a multidrug resistance-associated protein (MRP), a peptide transporter (PEPT), or a Na+ phosphate transporter (NPT)). In some embodiments, the functional RNA molecule modulates expression of a protein associated with decreased drug sensitivity, such as MAP kinase-activating death domain (MADD) protein, Smad3, or Smad4. In some embodiments, the functional RNA molecule is a siRNA molecule that decreases expression of a protein associated with decreased drug sensitivity, such as MAP kinase-activating death domain (MADD) protein, Smad3, or Smad4. In some embodiments, the functional RNA molecule with any of the above activities provides a chemotherapeutic effect.

The functional RNA molecule complexed with the small-molecule drug retains the functional activity of the functional RNA molecule. In some embodiments, the functional RNA molecule complexed with the small-molecule drug retains about 50% or more (such as about 60%, 700/%, 80%, 90%, 95%, or 100% or more) of the activity of the functional RNA molecule that is not complexed with the small-molecule drug.

Exemplary functional RNA molecules include siRNA, shRNA, miRNA, circularRNA (circRNA), rRNA, Piwi-interacting RNA (piRNA), toxic small RNA (tsRNA), or a ribozyme. In some embodiments, the RNA molecule is an antisense RNA molecule. The functional RNA molecule can include a nonfunctional component, which may be attached to the 5′ or 3′ end of the functional component of the functional RNA. In some embodiments, the functional RNA molecule is an anticancer agent, which can function, for example, by modulating gene expression, modulating an immune response by regulating one or more immune system checkpoint proteins, or regulating cytokine expression.

In some embodiments, the functional RNA molecule is double stranded. In some embodiments, the functional RNA molecule is single stranded and comprises at least one self-complementary region. A functional RNA molecule can comprise, for example, a stem-loop structure, wherein the stem portion of the RNA molecule includes the self-complementary region. The double-stranded functional RNA molecule need not be perfectly base paired, and in some embodiments comprises one or more bulges, loops, mismatches, or other secondary structure. In some embodiments, about 80% or more of the nucleotides are paired, about 85% or more of the nucleotides are paired, about 90% or more of the nucleotides are paired, about 95% of the nucleotides are paired, or about 100% of the nucleotides are paired.

In some embodiments, the functional RNA comprises one or more triphosphate 5′-ends, such as T7-transcribed RNA. The triphosphate 5′-end can trigger endogenous expression of type I interferons, which can further enhance the cancer cell death. In some embodiments, the RNA is synthetically produced or does not include one or more triphosphate 5′-ends.

In some embodiments, the functional RNA molecules are about 10-100 nucleotides in length, such as about 10-30, 20-40, 30-50, 40-60, 50-70, 60-80, 70-90, or 80-100 nucleotides in length. In some embodiments, the functional RNA molecules are about 25-35 nucleotides in length, such as about 25, 26, 27, 28, 29, 30, 31, 32, 33, 34, or 35 nucleotides in length. In some embodiments, the oligonucleotides are about 25-35 nucleotides in length, such as about 25, 26, 27, 28, 29, 30, 31, 32, 33, 34, or 35 nucleotides in length.

The functional RNA molecule is complexed with a small-molecule drug, such as a chemotherapeutic agent. Exemplary small-molecule drugs include anthracyclines (such as doxorubicin, daunorubicin, epirubicin, idarubicin, mitoxantrone, valrubicin) or alkylating or alkylating-like agents (such as carboplatin, carmustine, cisplatin, cyclophosphamide, melphalan, procarbazine, or thioTEPA). In some embodiments, the small-molecule compound is about 1500 Daltons or less, such as about 1000 Daltons, 900 Daltons, 800 Daltons, 700 Daltons, 600 Daltons, 500 Daltons, 400 Daltons, or 300 Daltons or less. In some embodiments, the small-molecule compound is about 100-1500 Daltons (such as about 100-200 Daltons, 200-300 Daltons, 300-400 Daltons, 400-500 Daltons, 500-600 Daltons, 600-700 Daltons, 700-800 Daltons, 800-900 Daltons, 900-1000 Daltons, 1000-1100 Daltons, 1100-1200 Daltons, 1200-1300 Daltons, 1300-1400 Daltons, or 1400-1500 Daltons).

In some embodiments, the small-molecule drug has a solubility (as measured in water, pH 7 at about 25° C.) of about 50 mg/mL or less (such as about 25 mg/mL, 10 mg/mL, 5 mg/mL, 2 mg/mL, 1 mg/mL, 0.5 mg/mL, 0.25 mg/mL, 0.1 mg/mL, 0.05 mg/mL, 0.025 mg/mL, 0.01 mg/mL, 0.005 mg/mL, 0.0025 mg/mL, or 0.001 mg/mL or less). In some embodiments, the small-molecule drug has a solubility (as measured in water, pH 7 at about 25° C.) of about 0.0001-50 mg/mL (such as about 0.0001-0.0005 mg/mL, 0.0005-0.001 mg/mL, 0.001-0.0025 mg/mL, 0.0025-0.005 mg/mL, 0.005-0.01 mg/mL, 0.01-0.025 mg/mL, 0.025-0.05 mg/mL, 0.05-0.1 mg/mL, 0.1-0.25 mg/mL, 0.25-0.5 mg/mL, 0.5-1 mg/mL, 1-2 mg/mL, 2-5 mg/mL, 5-10 mg/mL, 10-25 mg/mL, or 25-50 mg/mL).

In some embodiments, the molar ratio of the small-molecule drug to the functional RNA molecule in the therapeutic complex is about 60:1 or less, such as about 50:1, 40:1, 30:1, 20:1, 10:1, 5:1, 4:1, 3:1, 2:1, or 1:1 or less. In some embodiments, the molar ratio of the small-molecule drug to the functional RNA molecule in the therapeutic complex is between about 1:1 and about 60:1, such as about 1:1-10:1, 5:1-20:1, 10:1-30:1, 20:1-40:1, 30:1-50:1, or 40:1-60:1. In some embodiments, the molar ratio of the small-molecule drug to the functional RNA molecule in the therapeutic complex is about 1:1, 5:1, 10:1, 20:1, 30:1, 40:1, 50:1, or 60:1.

The small-molecule drug is complexed with the functional RNA molecule. In some embodiments, the small-molecule drug is complexed with the functional RNA molecule by electrostatic interactions, covalent bonds (such as a disulfide bond), or by intercalating the RNA. Complexing of the small-molecule drug to the functional RNA molecule is not sequence specific. In some embodiments, the functional RNA molecule is paired to a complementary RNA (such as in double-stranded RNA or a single-stranded RNA that has a self-complementary portion), which allows intercalation of the small-molecule drug between the paired bases. In some embodiments, average molar ratio of the small-molecule drug per paired base in the functional RNA molecule is about 1:1-1:120 (for example, about 1:2-1:120, 1:2-1:4, 1:4-1:8, 1:8-1:16, 1:16-1:32, 1:32-1:64, 1:64-1:100, or 1:100-1:120). It is understood that a base and its complement would be considered two paired bases when considering the molar ratio of small-molecule drug per paired base in the functional RNA molecule.

In some embodiments, there is provided a complex comprising a functional RNA molecule (such as a double-stranded siRNA molecule) complexed with a small-molecule drug. In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

In some embodiments, there is provided a liposome comprising a therapeutic complex, the therapeutic complex comprising a functional RNA molecule (such as a double-stranded siRNA molecule) complexed with a small-molecule drug. In some embodiments, the liposome comprises a targeting segment, which can target the liposome to a cell (such as a cancer cell). In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

The therapeutic complex can be formed by combining the functional RNA molecule with the small-molecule drug (such as a chemotherapeutic agent), which allows the small-molecule drug to bind or intercalate into the functional RNA molecule in a non-sequence specific manner. In some embodiments, the functional RNA molecule is a double stranded RNA molecule (or includes a double stranded segment), and the small-molecule drug intercalates into the double stranded functional RNA molecule. In some embodiments, the small-molecule drug and the functional RNA molecule are combined at a ratio (small molecule drug to functional RNA molecule) of about 60:1, 50:1, 40:1, 30:1, 20:1, 10:1, 5:1, 4:1, 3:1, 2:1, or 1:1 or less. In some embodiments, the small-molecule drug and the functional RNA molecule are combined at a ratio (small molecule drug to functional RNA molecule) between about 1:1 and about 60:1, such as about 1:1-10:1, 5:1-20:1, 10:1-30:1, 20:1-40:1, 30:1-50:1, or 40:1-60:1. In some embodiments, the small-molecule drug and the functional RNA molecule are combined at a ratio (small molecule drug to functional RNA molecule) of about 1:1, 1:10, 1:15, 1:20, 1:25, 1:30, 1:35, 1:40, 1:45, 1:50, 1:55, or 1:60.

Once the functional RNA molecule and the small-molecule drug are combined, the mixture can be incubated, which allows the small-molecule drug and the functional RNA molecule to form a complex, for example by allowing the small-molecule drug to intercalate into the functional RNA molecule. Unbound small-molecule drug can be separated from the complex, for example by centrifuging the complex using a filter membrane. The retentate will include the complex, and can be retained, while the filtrate includes unbound small-molecule drug.

In some embodiments, the therapeutic complex is sterilized, for example by using a sterile filter. In some embodiments, the therapeutic complex is lyophilized. In some embodiments, the lyophilized therapeutic complex is reconstituted prior to being formulated for administration or formulated with a carrier (e.g., liposome or nanoparticle).

The formed therapeutic complex can be loaded into a carrier, such as a liposome or a nanoparticle. Accordingly, in some embodiments, there is provided a composition comprising a liposome comprising a therapeutic complex, wherein the therapeutic complex comprises a functional RNA and a small-molecule drug. The liposome can include cationic lipids (such as lipofectamine), which can bind to the negative charges of the functional RNA molecule of the therapeutic complex. In some embodiments, the therapeutic complex is loaded into a nanoparticle, for example a nanoparticle that includes a carrier polypeptide comprising a cell-penetrating segment and an oligonucleotide-binding segment. In some embodiments, the carrier is a targeted carrier that includes a targeting segment, such as an antibody or a receptor binding domain.

The therapeutic complex including the functional RNA and the small-molecule drug can be useful for killing a cell (such as a cancer cell), inducing apoptosis of a cell (such as a cancer cell), or treating cancer in a patient.

In some embodiments, there is a method of delivering a therapeutic complex to a cell (such as a cancer cell), comprising transfecting the cell with a complex comprising a functional RNA molecule (such as a double-stranded siRNA molecule) and a small-molecule drug. In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

In some embodiments, there is a method of delivering a therapeutic complex to a cell (such as a cancer cell), comprising contacting the cell with a composition comprising liposomes comprising the therapeutic complex, the therapeutic complex comprising a functional RNA molecule (such as a double-stranded siRNA molecule) complexed with a small-molecule drug. In some embodiments, the liposome comprises a targeting segment, which can target the liposome to the cell. In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

In some embodiments, there is provided a method of killing a cell (such as a cancer cell), comprising transfecting the cell with a complex comprising a functional RNA molecule and a small-molecule drug (such as a chemotherapeutic drug). In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

In some embodiments, there is provided a method of killing a cell (such as a cancer cell), comprising contacting the cell with a composition comprising liposomes comprising the therapeutic complex, the therapeutic complex comprising a functional RNA molecule (such as a double-stranded siRNA molecule) complexed with a small-molecule drug. In some embodiments, the liposome comprises a targeting segment, which can target the liposome to the cell. In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

In some embodiments, there is provided a method of inducing apoptosis of a cell (such as a cancer cell), comprising transfecting the cell with a complex comprising a functional RNA molecule and a small-molecule drug. In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

In some embodiments, there is provided a method of inducing apoptosis of a cell (such as a cancer cell), comprising contacting the cell with a composition comprising liposomes comprising the therapeutic complex, the therapeutic complex comprising a functional RNA molecule (such as a double-stranded siRNA molecule) complexed with a small-molecule drug. In some embodiments, the liposome comprises a targeting segment, which can target the liposome to the cell. In some embodiments, the functional RNA molecule modulates expression of one or more proteins. In some embodiments, the functional RNA molecule includes at least one complementary region or is a double-stranded RNA molecule. In some embodiments, the small-molecule drug intercalates into the functional RNA molecule. In some embodiments, the molar ratio of the RNA molecule to the small-molecule drug is about 1:10 to about 1:60. In some embodiments, the small-molecule drug is a chemotherapeutic agent, such as an anthracycline (for example, doxorubicin) or an alkylating or an alkylating-like agent.

In some embodiments, there is provided a method of treating cancer in a subject, comprising administering to the subject an effective amount of a complex comprising a functional RNA molecule and a small-molecule chemotherapeutic drug. In some embodiments, the functional RNA molecule modulates expression of one or more proteins. In some embodiments, the functional RNA molecule includes at least one complementary region or is a double-stranded RNA molecule. In some embodiments, the small-molecule chemotherapeutic drug intercalates into the functional RNA molecule. In some embodiments, the molar ratio of the RNA molecule to the small-molecule drug is about 1:10 to about 1:60. In some embodiments, the small-molecule chemotherapeutic drug is an anthracycline (for example, doxorubicin), an alkylating agent, or an alkylating-like agent. In some embodiments, there is provided a therapeutic complex for use in the treatment of cancer, the therapeutic complex comprising a functional RNA molecule complexed with a small-molecule chemotherapeutic drug. Further provided herein is a therapeutic complex for use in the manufacture of a medicament for the treatment of cancer, the therapeutic complex comprising a functional RNA molecule complexed with a small-molecule chemotherapeutic drug.

In some embodiments, there is provided a method of treating cancer in a subject, comprising administering to the subject an effective amount of a composition comprising liposomes comprising a therapeutic complex, the therapeutic complex comprising a functional RNA molecule complexed with a small-molecule chemotherapeutic drug. In some embodiments, the functional RNA molecule modulates expression of one or more proteins. In some embodiments, the functional RNA molecule includes at least one complementary region or is a double-stranded RNA molecule. In some embodiments, the small-molecule chemotherapeutic drug intercalates into the functional RNA molecule. In some embodiments, the molar ratio of the RNA molecule to the small-molecule drug is about 1:10 to about 1:60. In some embodiments, the small-molecule chemotherapeutic drug is an anthracycline (for example, doxorubicin), an alkylating agent, or an alkylating-like agent. In some embodiments, there is provided a liposome for use in the treatment of cancer, the liposome comprising a therapeutic complex comprising a functional RNA molecule complexed with a small-molecule chemotherapeutic drug. Further provided herein is a composition comprising liposomes for use in the manufacture of a medicament for the treatment of cancer, the liposomes comprising a therapeutic complex comprising a functional RNA molecule complexed with a small-molecule chemotherapeutic drug.

### Nanoparticle Compositions

The nanoparticle compositions described herein comprises a carrier polypeptide, which comprises a cell-penetrating segment and an oligonucleotide-binding segment. In some embodiments, the nanoparticle compositions described herein comprise a carrier polypeptide, which comprises a cell-targeting segment, a cell-penetrating segment, and an oligonucleotide-binding segment. The nanoparticles further comprise a functional RNA molecule complexed with a small-molecule drug. The functional RNA molecule can bind the oligonucleotide-binding segment of the carrier polypeptide. Upon binding of the carrier polypeptide to the functional RNA molecule, the nanoparticles spontaneously form.

The functional RNA molecule can provide a biological function, such as causing inhibition of protein expression (for example, through an RNAi pathway), an increase in protein expression (for example, through the use of mRNA as the functional RNA molecule), or altered expression of one or more cytokines (such as a type I interferon (e.g., IFN-α, INF-β), IL-6, or IL-8)). In some embodiments, the functional RNA molecule is an anti-HER2 siRNA. In some embodiments, the functional RNA molecule modulates expression of an immune system checkpoint protein (e.g., programmed cell death protein ligand 1 (PD-L1), or programmed cell death protein 1 (PD-1), or cytotoxic T-lymphocyte-associated protein 4 (CTLA-4)) expressed by a tumor cell. In some embodiments, the functional RNA molecule is a siRNA molecule that decreases expression of an immune system checkpoint protein. In some embodiments, the functional RNA molecule modulates expression of a protein associated with drug efflux or drug resistance (such as a monocarboxylate transporter (MCT), a multiple drug resistance protein (MDR), a P-glycoprotein, a multidrug resistance-associated protein (MRP), a peptide transporter (PEPT), or a Na+ phosphate transporter (NPT)). In some embodiments, the functional RNA molecule is an siRNA molecule that decreases expression of a protein associated with drug efflux or drug resistance (such as a monocarboxylate transporter (MCT), a multiple drug resistance protein (MDR), a P-glycoprotein, a multidrug resistance-associated protein (MRP), a peptide transporter (PEPT), or a Na+ phosphate transporter (NPT)). In some embodiments, the functional RNA molecule modulates expression of a protein associated with decreased drug sensitivity, such as MAP kinase-activating death domain (MADD) protein, Smad3, or Smad4. In some embodiments, the functional RNA molecule is a siRNA molecule that decreases expression of a protein associated with decreased drug sensitivity, such as MAP kinase-activating death domain (MADD) protein, Smad3, or Smad4. In some embodiments, the functional RNA molecule with any of the above activities provides a chemotherapeutic effect.

Exemplary functional RNA molecules include siRNA, shRNA, miRNA, circularRNA (circRNA), rRNA, Piwi-interacting RNA (piRNA), toxic small RNA (tsRNA), or a ribozyme. In some embodiments, the RNA molecule is an antisense RNA molecule. The functional RNA molecule can include a nonfunctional component, which may be attached to the 5′ or 3′ end of the functional component of the functional RNA. In some embodiments, the functional RNA molecule is an anticancer agent, which can function, for example, by modulating gene expression or regulating cytokine expression.

The functional RNA molecule complexed with the small-molecule drug retains the functional activity of the functional RNA molecule. In some embodiments, the functional RNA molecule complexed with the small-molecule drug retains about 50% or more (such as about 60%, 70%, 80%, 90%, 95%, or 100% or more) of the activity of the functional RNA molecule that is not complexed with the small-molecule drug.

In some embodiments, the functional RNA molecule is double stranded. In some embodiments, the functional RNA molecule is single stranded and comprises at least one self-complementary region. A functional RNA molecule can comprise, for example, a stem-loop structure, wherein the stem portion of the RNA molecule includes the self-complementary region. The double-stranded functional RNA molecule need not be perfectly base paired, and in some embodiments comprises one or more bulges, loops, mismatches, or other secondary structure. In some embodiments, about 80% or more of the nucleotides are paired, about 85% or more of the nucleotides are paired, about 90% or more of the nucleotides are paired, about 95% of the nucleotides are paired, or about 100% of the nucleotides are paired.

In some embodiments, the functional RNA comprises one or more triphosphate 5′-ends, such as T7-transcribed RNA. The triphosphate 5′-end can trigger endogenous expression of type I interferons, which can further enhance the cancer cell death. In some embodiments, the RNA is synthetically produced or does not include one or more triphosphate 5′-ends.

In some embodiments, the functional RNA molecules are about 10 nucleotides in length to about 100 nucleotides in length, such as about 10-100 nucleotides in length, such as about 10-30, 20-40, 30-50, 40-60, 50-70, 60-80, 70-90, or 80-100 nucleotides in length. In some embodiments, the oligonucleotides are about 25-35 nucleotides in length, such as about 25, 26, 27, 28, 29, 30, 31, 32, 33, 34, or 35 nucleotides in length. In some embodiments, the oligonucleotides are about 15-25 nucleotides in length, such as about 15, 16, 17, 18, 19, 20, 21, 22, 23, 24, or 25 nucleotides in length.

The functional RNA molecule in the nanoparticle is complexed with a small-molecule drug, such as a chemotherapeutic agent. The small-molecule drug can complex with the functional RNA molecule, for example, by electrostatic interactions or by intercalating in the functional RNA molecule. Exemplary small-molecule drugs include anthracyclines (such as doxorubicin, daunorubicin, epirubicin, idarubicin, mitoxantrone, valrubicin) or alkylating or alkylating-like agents (such as carboplatin, carmustine, cisplatin, cyclophosphamide, melphalan, procarbazine, or thiotepa). In some embodiments, the small-molecule compound is about 1500 Daltons or less, such as about 1000 Daltons, 900 Daltons, 800 Daltons, 700 Daltons, 600 Daltons, 500 Daltons, 400 Daltons, or 300 Daltons or less. In some embodiments, the small-molecule compound is about 100-1500 Daltons (such as about 100-200 Daltons, 200-300 Daltons, 300-400 Daltons, 400-500 Daltons, 500-600 Daltons, 600-700 Daltons, 700-800 Daltons, 800-900 Daltons, 900-1000 Daltons, 1000-1100 Daltons, 1100-1200 Daltons, 1200-1300 Daltons, 1300-1400 Daltons, or 1400-1500 Daltons).

In some embodiments, the small-molecule drug has a solubility (as measured in water, pH 7 at about 25° C.) of about 50 mg/mL or less (such as about 25 mg/mL, 10 mg/mL, 5 mg/mL, 2 mg/mL, 1 mg/mL, 0.5 mg/mL, 0.25 mg/mL, 0.1 mg/mL, 0.05 mg/mL, 0.025 mg/mL, 0.01 mg/mL, 0.005 mg/mL, 0.0025 mg/mL, or 0.001 mg/mL or less). In some embodiments, the small-molecule drug has a solubility (as measured in water, pH 7 at about 25° C.) of about 0.0001-50 mg/mL (such as about 0.0001-0.0005 mg/mL, 0.0005-0.001 mg/mL, 0.001-0.0025 mg/mL, 0.0025-0.005 mg/mL, 0.005-0.01 mg/mL, 0.01-0.025 mg/mL, 0.025-0.05 mg/mL, 0.05-0.1 mg/mL, 0.1-0.25 mg/mL, 0.25-0.5 mg/mL, 0.5-1 mg/mL, 1-2 mg/mL, 2-5 mg/mL, 5-10 mg/mL, 10-25 mg/mL, or 25-50 mg/mL).

In some embodiments, the molar ratio of the small-molecule drug to the functional RNA molecule in the therapeutic complex is about 60:1 or less, such as about 50:1, 40:1, 30:1, 20:1, 10:1, 5:1, 4:1, 3:1, 2:1, or 1:1 or less. In some embodiments, the molar ratio of the small-molecule drug to the functional RNA molecule in the therapeutic complex is between about 1:1 and about 60:1, such as about 1:1-10:1, 5:1-20:1, 10:1-30:1, 20:1-40:1, 30:1-50:1, or 40:1-60:1. In some embodiments, the molar ratio of the small-molecule drug to the functional RNA molecule in the therapeutic complex is about 1:1, 5:1, 10:1, 20:1, 30:1, 40:1, 50:1, or 60:1.

The small-molecule drug is complexed with the functional RNA molecule. In some embodiments, the small-molecule drug is complexed with the functional RNA molecule by electrostatic interactions, covalent bonds (such as a disulfide bond), or by intercalating the RNA. For example, the functional RNA can be paired to a complementary RNA (such as in double-stranded RNA or a single-stranded RNA that has a self-complementary portion), which allows intercalation of the small-molecule drug between the paired bases. In some embodiments, average molar ratio of the small-molecule drug per paired base in the functional RNA molecule is about 1:1-1:120 (for example, about 1:2-1:120, 1:2-1:4, 1:4-1:8, 1:8-1:16, 1:16-1:32, 1:32-1:64, 1:64-1:100, or 1:100-1:120). It is understood that a base and its complement would be considered two paired bases when considering the molar ratio of small-molecule drug per paired base in the functional RNA molecule.

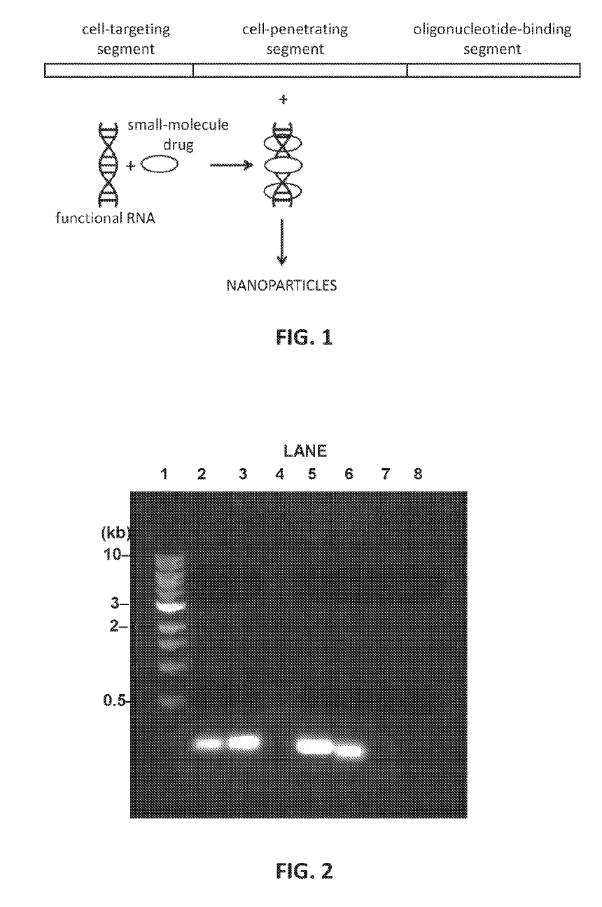

The cell-targeting segment, the cell-penetrating segment, and the oligonucleotide-binding segment are fused together in a single carrier polypeptide. The segments described herein are modular, and can be combined in various combinations. That is, a carrier polypeptide can comprise any of the described cell-targeting segments, the cell-penetrating segments, or the oligonucleotide-binding segments. FIG. 1 illustrates a carrier peptide with a cell-targeting segment, a cell-penetrating segment, and an oligonucleotide-binding segment. As further shown in FIG. 1, combining the carrier peptide with the functional RNA molecule results in the formation of nanoparticles. Optionally, the functional RNA molecule is pre-bound to a small-molecule drug prior to forming the nanoparticles.

The nanoparticles can be formed by combining the carrier polypeptide with a functional RNA molecule. In some embodiments, the carrier polypeptide is combined with the functional RNA molecule at a molar ratio of about 8:1 or less (for example, about 3:1-8:1, 3:1-3.5:1, 3.5:1-4:1, 4:1-4.5:1, 4.5:1-5:1, 5:1-5.5:1, 5.5:1-6:1, 6:1-6.5:1, 6.5:1-7:1, 7:1-7.5:1, or 7.5:1-8:1), thereby forming a nanoparticle composition. In some embodiments, the carrier polypeptide is combined with the functional RNA molecule at a molar ratio of about 4:1, 4.5:1, 5:1, 5.5:1, 6:1, 6.5:1, 7:1, 7.5:1, or 8:1. Thus, in some embodiments, the nanoparticle composition comprises carrier polypeptides and the functional RNA molecule at a molar ratio of about 8:1 or less (for example, about 3:1-8:1, 3:1-3.5:1, 3.5:1-4:1, 4:1-4.5:1, 4.5:1-5:1, 5:1-5.5:1, 5.5:1-6:1, 6:1-6.5:1, 6.5:1-7:1, 7:1-7.5:1, or 7.5:1-8:1). In some embodiments, the carrier polypeptide is combined with the functional RNA molecule at a molar ratio of about 4:1, 4.5:1, 5:1, 5.5:1, 6:1, 6.5:1, 7:1, 7.5:1, or 8:1.

In some embodiments, the nanoparticle composition comprises nanoparticles with a homogenous molar ratio of carrier polypeptides to functional RNA molecule. In some embodiments, the nanoparticles comprise carrier polypeptides and functional RNA molecules at a molar ratio of about 8:1, 7:1, 6:1, 5:1, 4:1, or 3:1.

In some embodiments the nanoparticles in the nanoparticle composition have an average size of about 100 nm or less (such as about 90 nm, 80 nm, 70 nm, 60 nm, 50 nm, or 40 nm or less). In some embodiments, nanoparticles have an average size between about 30 nm and about 100 nm (such as about 30-40 nm, 40-50 nm, 50-60 nm, 60-70 nm, 70-80 nm, 80-90 nm or 90-100 nm.

The cell-targeting segment can bind to a target molecule present on the surface of a cell. Binding of the molecule by the cell-targeting segment allows the nanoparticle to be targeted to the cell. Thus, the targeted molecule present on the cell can depend on the targeted cell. In some embodiments, the targeted molecule is an antigen, such as a cancer antigen. In some embodiments, the cancer cell exhibits upregulated expression of the target molecule. The upregulated expression may be for example, an increase of about 10%, 20%, 30%, 40%, 40%, 50%, 60%, 70%, 80%, 90%, 100% or more. In some embodiments, the targeted molecule is a cell surface receptor, such as HER3 or c-MET. In some embodiments, the cell-targeting segment binds to of 4-IBB, 5T4, adenocarcinoma antigen, alpha-fetoprotein, BAFF, C242 antigen, CA-125, carbonic anhydrase 9 (CA-IX), c-MET, CCR4, CD152, CD19, CD20, CD200, CD22, CD221, CD23 (IgE receptor), CD28, CD30 (TNFRSF8), CD33, CD4, CD40, CD44v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, hepatocyte growth factor (HGF), human scatter factor receptor kinase, IGF-1 receptor, IGF-1, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-IC, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, Internalin B, bacterial invasin (Inv) protein, or a fragment thereof.

In some embodiments, the cell-targeting segment comprises an antibody, an antibody fragment (such as a Fab fragment, a F(ab′)₂ fragment, a Fab′ fragment, or a single-chain variable (scFv) fragment) a cytokine, or a receptor ligand.

In some embodiment, the cell-targeting segment comprises a ligand that specifically binds to a receptor expressed on the surface of a cell. Exemplary ligands include a heregulin sequence (or a variant thereof) or an Internalin B sequence (or a variant thereof). The heregulin sequence can be, for example, a heregulin-α sequence, such as a receptor binding domain of heregulin-α. The receptor binding domain of heregulin-α includes an IG-like domain and an EGF-like domain. The ligand variants retain specific binding for the targeted molecule. Heregulin (which can be referred to as “Her”) can specifically bind to HER3. SEQ ID NO: 2 is an exemplary wild-type Her sequence, which includes the Ig-like domain and the EGF-like domain of the receptor binding sequence of heregulin-α. Internalin B can specifically bind to c-MET, and can also be referred to as “InlB”.

In some embodiments, the cell targeted by the cell-targeting segment is a mammalian cell, such as a human cell. In some embodiments, the cell is a diseased cell, such as a cancer cell. In some embodiment, the cell is a HER3+ cancer cell or a c-MET+ cancer cell. In some embodiment, the cell is a head and neck cancer cell, a pancreatic cancer cell, a breast cancer cell, a glial cancer cell, an ovarian cancer cell, a cervical cancer cell, a gastric cancer cell, a skin cancer cell, a colon cancer cell, a rectal cancer cell, a lung cancer cell, a kidney cancer cell, a prostate cancer cell, or a thyroid cancer cell. The cell-targeting segment can bind a molecule present on the surface of the targeted cell, which targets the nanoparticle to the targeted cell.

The cell-penetrating segment of the carrier polypeptide facilitates entry of the nanoparticle into the cell targeted by the cell-targeting segment. In some embodiments, the cell-penetrating segment comprises (and, in some embodiments, is) a penton base (“PB”) protein, or a variant thereof. By way of example, in some embodiments, the cell-penetrating segment comprises (and, in some embodiments, is) the adenovirus serotype 5 (Ad5) penton base protein. In some embodiments, the cell-targeting segment comprises (and, in some embodiments, is) a penton base protein with an amino acid variation or deletion. The amino acid variation can be a conservative mutation. In some embodiments, the cell-targeting segment is a truncated penton base protein.

The cell-penetrating segment can comprise one or more variants that enhance subcellular localization of the carrier polypeptide. For example, in some embodiments, the cell-penetrating segment comprises one or more variants which cause the carrier polypeptide to preferentially localize in the cytoplasm or the nucleus. In embodiments, where the carrier polypeptide is bound to a functional RNA molecule (which is itself complexed to a small-molecule drug), the variant cell-penetrating segment preferentially localizes the functional RNA molecule and small-molecule drug to the cytoplasm or the nucleus. Preferential subcellular localization can be particular beneficial for certain small-molecule drugs. For example, many chemotherapeutic agents function by binding to DNA localized in the cancer cell nucleus. By preferentially targeting the nucleus, the associated drug is concentrated at the location it functions. Other small-molecule drugs may function in the cytoplasm, and preferentially targeting to the cytoplasm can enhance drug potency.

Exemplary cell-penetrating segment mutations that enhance subcellular localization are discussed in WO 2014/022811. The Leu60Trp mutation in the penton base protein has been shown to preferentially localize to the cytoplasm of the cell. Thus, in some embodiments, the cell-penetrating segment is a penton base protein comprising the Leu60Trp mutation. The Lys375Glu, Val449Met, and Pro469Ser mutations have been shown to preferentially localize to the nucleus of the cell. Thus, in some embodiments, the cell-penetrating segment is a penton base protein comprising a Lys375Glu, Val449Met, or Pro469Ser mutations. In some embodiments, the cell-penetrating segment is a penton base protein comprising the Lys375Glu, Val449Met, and Pro469Ser mutations. Amino acid numbering is made in reference to the wild-type penton base polypeptide of SEQ ID NO: 1.

The oligonucleotide-binding segment binds the functional RNA molecule component of the nanoparticle. The oligonucleotide-binding segment can bind the functional RNA molecule, for example, through electrostatic bonds, hydrogen bonds, or ionic bonds. In some embodiments, the oligonucleotide-binding segment is an RNA binding domain or a double-stranded RNA binding domain. In some embodiments, the oligonucleotide-binding segment is a cationic (i.e., positively charged) domain. In some embodiments, the oligonucleotide binding domain comprises is a polylysine sequence. In some embodiments, the oligonucleotide-binding segment is between about 3 and about 30 amino acids in length, such as between about 3 and about 10, between about 5 and about 15, between about 10 and about 20, between about 15 and about 25, or between about 20 and about 30 amino acids in length. In one exemplary embodiment, the oligonucleotide-binding segment comprises (and, in some embodiments, is) a decalysine (that is, ten sequential lysine amino acids, or “K10,” as shown in SEQ ID NO: 4).

Exemplary carrier polypeptides comprises Her (or a variant thereof), a penton base (or a variant thereof), and a positively charged oligonucleotide-binding segment. In some embodiments, the carrier polypeptide comprises Her, a penton base segment, and a polylysine oligonucleotide-binding segment. In some embodiment, the carrier polypeptide comprises Her, a penton base segment, and a decalysine oligonucleotide-binding segment, for example HerPBK10 (SEQ ID NO: 3). Other exemplary embodiments comprise InlB, a penton base (or a variant thereof), and a positively charged oligonucleotide-binding segment, such as InlBPBK10.

In some embodiments, the nanoparticles are about 50 nm or less in diameter (such as about 45 nm, 40 nm, 35 nm, or 30 nm or less, as measured by dynamic light scattering. In some embodiments, the nanoparticles are about 25-50 nm, 25-30 nm, 30-35 nm, 35-40 nm, or 45-50 nm in diameter, as measured by dynamic light scattering.

In one aspect, there is provided a composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment. In some embodiments, the functional RNA molecule is about 10 nucleotides to about 100 nucleotides in length. In some embodiments, the molar ratio of the functional RNA molecule to the small-molecule drug in the nanoparticle composition is about 1:1 to about 1:60. In some embodiments, the molar ratio of the carrier polypeptide to the functional RNA molecule in the composition is about 3:1 to about 8:1 (such as about 4:1). In some embodiments, the cell-targeting segment binds a mammalian cell, which may be a diseased cell (such as a cancer cell). In some embodiments, the cell-targeting segment binds a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less).

In another aspect, there is provided a composition comprising nanoparticles comprising a carrier polypeptide and a small-molecule drug intercalated into a functional RNA molecule, wherein the carrier polypeptide comprises a cell-targeting segment, a cell-penetrating segment, and an oligonucleotide-binding segment. In some embodiments, the functional RNA molecule is about 10 nucleotides to about 100 nucleotides in length. In some embodiments, the molar ratio of the functional RNA molecule to the small-molecule drug in the nanoparticle composition is about 1:1 to about 1:60. In some embodiments, the molar ratio of the carrier polypeptide to the functional RNA molecule in the composition is about 3:1 to about 8:1 (such as about 4:1). In some embodiments, the cell-targeting segment binds a mammalian cell, which may be a diseased cell (such as a cancer cell). In some embodiments, the cell-targeting segment binds a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less).

In another aspect, there is provided a composition comprising nanoparticles comprising a carrier polypeptide and a small-molecule drug intercalated into a double-stranded siRNA molecule, wherein the carrier polypeptide comprises a cell-targeting segment, a cell-penetrating segment, and an oligonucleotide-binding segment. In some embodiments, the siRNA molecule is about 10 nucleotides to about 100 nucleotides in length. In some embodiments, the molar ratio of the siRNA molecule to the small-molecule drug in the nanoparticle composition is about 1:1 to about 1:60. In some embodiments, the molar ratio of the carrier polypeptide to the siRNA molecule in the composition is about 3:1 to about 8:1 (such as about 4:1). In some embodiments, the cell-targeting segment binds a mammalian cell, which may be a diseased cell (such as a cancer cell). In some embodiments, the cell-targeting segment binds a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less).

In another aspect, there is provided a composition comprising nanoparticles comprising a carrier polypeptide and a small-molecule drug intercalated into a double-stranded siRNA molecule, wherein the carrier polypeptide comprises a cell-targeting segment, a cell-penetrating segment, and an oligonucleotide-binding segment, and wherein the siRNA comprises at least one 5′-triphosphate end. In some embodiments, the siRNA molecule is about 10 nucleotides to about 100 nucleotides in length. In some embodiments, the molar ratio of the siRNA molecule to the small-molecule drug in the nanoparticle composition is about 1:1 to about 1:60. In some embodiments, the molar ratio of the carrier polypeptide to the siRNA molecule in the composition is about 3:1 to about 8:1 (such as about 4:1). In some embodiments, the cell-targeting segment binds a mammalian cell, which may be a diseased cell (such as a cancer cell). In some embodiments, the cell-targeting segment binds a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less).

In another aspect, there is provided a composition comprising nanoparticles comprising a carrier polypeptide and a small-molecule chemotherapeutic agent intercalated into a double-stranded siRNA molecule, wherein the carrier polypeptide comprises a cell-targeting segment, a cell-penetrating segment, and an oligonucleotide-binding segment, and wherein the siRNA comprises at least one 5′-triphosphate end. In some embodiments, the siRNA molecule is about 10 nucleotides to about 100 nucleotides in length. In some embodiments, the molar ratio of the siRNA molecule to the chemotherapeutic agent in the nanoparticle composition is about 1:1 to about 1:60. In some embodiments, the molar ratio of the carrier polypeptide to the siRNA molecule in the composition is about 3:1 to about 8:1 (such as about 4:1). In some embodiments, the cell-targeting segment binds a mammalian cell, which may be a diseased cell (such as a cancer cell). In some embodiments, the cell-targeting segment binds a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less). In some embodiments, the chemotherapeutic agent is an anthracycline (such as doxorubicin) or an alkylating agent or an alkylating-like agent.

In another aspect, there is provided a composition comprising nanoparticles comprising a carrier polypeptide and a small-molecule chemotherapeutic agent intercalated into a double-stranded siRNA molecule, wherein the carrier polypeptide comprises a cell-targeting segment, a cell-penetrating segment, and an oligonucleotide-binding segment, wherein the siRNA comprises at least one 5′-triphosphate end, and wherein the cell-targeting segment targets a HER3+ cancer cell. In some embodiments, the siRNA molecule is about 10 nucleotides to about 100 nucleotides in length. In some embodiments, the molar ratio of the siRNA molecule to the chemotherapeutic agent in the nanoparticle composition is about 1:1 to about 1:60. In some embodiments, the molar ratio of the carrier polypeptide to the siRNA molecule in the composition is about 3:1 to about 8:1 (such as about 4:1). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less). In some embodiments, the chemotherapeutic agent is an anthracycline (such as doxorubicin) or an alkylating agent or an alkylating-like agent. In some embodiments, the cell-targeting segment comprises a heregulin sequence or a variant thereof.

In another aspect, there is provided a composition comprising nanoparticles comprising a carrier polypeptide and a small-molecule chemotherapeutic agent intercalated into a double-stranded siRNA molecule, wherein the carrier polypeptide comprises a cell-targeting segment, a cell-penetrating segment, and an oligonucleotide-binding segment, wherein the siRNA comprises at least one 5′-triphosphate end, and wherein the cell-targeting segment targets a c-MET+ cancer cell. In some embodiments, the siRNA molecule is about 10 nucleotides to about 100 nucleotides in length. In some embodiments, the molar ratio of the siRNA molecule to the chemotherapeutic agent in the nanoparticle composition is about 1:1 to about 1:60. In some embodiments, the molar ratio of the carrier polypeptide to the siRNA molecule in the composition is about 3:1 to about 8:1 (such as about 4:1). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less). In some embodiments, the chemotherapeutic agent is an anthracycline (such as doxorubicin) or an alkylating agent or an alkylating-like agent. In some embodiments, the cell-targeting segment comprises an Internalin B sequence or a variant thereof.

In another aspect, there is provided a composition comprising nanoparticles comprising HerPBK10 and a small-molecule chemotherapeutic agent intercalated into a double-stranded siRNA molecule, wherein the siRNA comprises at least one 5′-triphosphate end. In some embodiments, the siRNA molecule is about 10 nucleotides to about 100 nucleotides in length. In some embodiments, the molar ratio of the siRNA molecule to the chemotherapeutic agent in the nanoparticle composition is about 1:1 to about 1:60. In some embodiments, the molar ratio of the carrier polypeptide to the siRNA molecule in the composition is about 3:1 to about 8:1 (such as about 4:1). In some embodiments, the average size of the nanoparticles in the composition is about 100 nm or less (such as about 60 nm or less, or about 50 nm or less). In some embodiments, the chemotherapeutic agent is an anthracycline (such as doxorubicin) or an alkylating agent or an alkylating-like agent.

### Production of Nanoparticles

The nanoparticles described herein can be produced by combining a plurality of carrier polypeptides with functional RNA molecules and small-molecule drugs. In some embodiments, the carrier polypeptides, the functional RNA molecules, and the small-molecule drug are incubated together to form the nanoparticles. In some embodiments, the functional RNA molecules are pre-incubated with the small-molecule drug prior to being combined with the carrier polypeptides. Upon combining the carrier polypeptide and the functional RNA molecules, the nanoparticles spontaneously assemble.

In some embodiments, single-stranded, complementary (or partially complementary or self-complementary) RNA molecules are annealed to form the functional RNA molecules used to form the nanoparticles. Annealing of the oligonucleotides can occur, for example, by combining RNA molecules, heating the RNA molecules (for example, to about 80° C. or higher), and cooling the mixture (for example, at about room temperature).

The small-molecule drug is bound to the functional RNA molecule by combining the small-molecule drug and the functional RNA molecules. In some embodiments, the small-molecule drug and the functional RNA molecules are combined at a molar ratio of about 60:1, 50:1, 40:1, 30:1, 20:1, 10:1, 5:1, 4:1, 3:1, 2:1, or 1:1 or less. In some embodiments, the small-molecule drug and the functional RNA molecules are combined at a molar ratio between about 1:1 and about 60:1, such as about 1:1-10:1, 5:1-20:1, 10:1-30:1, 20:1-40:1, 30:1-50:1, or 40:1-60:1. In some embodiments, the small-molecule drug and the functional RNA molecules are combined at a molar ratio of about 1:1, 1:10, 1:15, 1:20, 1:25, 1:30, 1:35, 1:40, 1:45, 1:50, 1:55, or 1:60. The small-molecule drug can be mixed with the RNA molecules prior to, during, or after the annealing process. Once the small-molecule drug and the functional RNA molecules are combined, the small-molecule drug binds to the functional RNA molecule, for example by intercalating into functional RNA molecule or by electrostatic interactions.

The functional RNA molecule and the small-molecule drug (which may be pre-complexed together) are combined with the carrier polypeptide to form the nanoparticles. In some embodiments, the carrier peptide and the functional RNA molecule are combined at a molar ratio of about 8:1 or less (for example, about 3:1-8:1, 3:1-3.5:1, 3.5:1-4:1, 4:1-4.5:1, 4.5:1-5:1, 5:1-5.5:1, 5.5:1-6:1, 6:1-6.5:1, 6.5:1-7:1, 7:1-7.5:1, or 7.5:1-8:1). In some embodiments, the carrier peptide and the functional RNA molecule are combined at a molar ratio of about 4:1, 4.5:1, 5:1, 5.5:1, 6:1, 6.5:1, 7:1, 7.5:1, or 8:1). In some embodiments, the carrier polypeptide and the functional RNA molecule are incubated at about 4° C. to about 22° C., such as about 4-15° C., or 4-10° C. In some embodiments, the carrier polypeptide and the functional RNA molecule incubate for less than about 30 minutes, about 30 minutes or more, about 1 hour or more, or about 2 hours or more. After combining the carrier polypeptide with the functional RNA molecule, the nanoparticles spontaneously form.

In some embodiments, excess oligonucleotide, small-molecule drug, or carrier polypeptide are removed from the composition comprising the nanoparticles. For example, in some embodiments, the nanoparticle composition is subjected to a purification step, such as size exclusion chromatography. In some embodiments, the unbound components are separated from the nanoparticles by ultracentrifugation. For example, in some embodiments, the composition is added to a centrifugal filter with a molecular weight cutoff of about 100 kD, 80 kD, 70 kD, 60 kD, 50 kD, 40 kD, 30 kD, 20 kD, 10 kD, or 5 kD or less.

Optionally, the resulting nanoparticle composition is subjected to buffer exchange, for example by dialysis, ultracentrifugation, or tangential flow filtration. In some embodiments, the nanoparticles are concentrated, for example by ultracentrifugation.

The nanoparticle composition can undergo further processing steps. For example in some embodiments, the nanoparticle composition is sterilized, for example by sterile filtration. In some embodiments, the nanoparticle composition is dispensed into a vial (which may then be sealed). In some embodiments, the nanoparticle composition is lyophilized, thereby forming a dry nanoparticle composition. In some embodiments, the nanoparticle composition is formulated to form a pharmaceutical composition, for example by adding one or more pharmaceutically acceptable excipients.

In one aspect, there is provided a method of making a nanoparticle composition comprising combining a carrier polypeptide, a functional RNA molecule, and a small-molecule drug, wherein the carrier polypeptide comprises a cell-targeting segment, a cell-penetrating segment, and an oligonucleotide-binding segment. In some embodiments, the small-molecule drug intercalates the RNA molecule. In some embodiments, the nanoparticle composition is sterile filtered or lyophilized. In some embodiments, the functional RNA molecule is about 10 nucleotides to about 100 nucleotides in length. In some embodiments, the functional RNA molecule and the small-molecule drug are provided at a molar ratio of about 1:1 to about 1:60. In some embodiments, the carrier polypeptide and the functional RNA molecule are provided at a molar ratio of about 3:1 to about 8:1 (such as about 4:1). In some embodiments, the cell-targeting segment is configured to bind to a mammalian cell, which may be a diseased cell (such as a cancer cell). In some embodiments, the cell-targeting segment is configured to bind to a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the resulting nanoparticles is about 100 nm or less (such as about 60 nm or less, or about 50 nm or less).

In another aspect, there is provided a method of making a nanoparticle composition comprising combining a functional RNA molecule with the small-molecule drug to complex the drug to the RNA molecule; and combining a carrier polypeptide with the RNA molecule complexed with the small-molecule drug, wherein the carrier polypeptide comprises a cell-targeting segment, a cell-penetrating segment, and an oligonucleotide-binding segment. In some embodiments, the small-molecule drug intercalates the RNA molecule. In some embodiments, the nanoparticle composition is sterile filtered or lyophilized. In some embodiments, the functional RNA molecule is about 10 nucleotides to about 100 nucleotides in length. In some embodiments, the functional RNA molecule and the small-molecule drug are provided at a molar ratio of about 1:1 to about 1:60. In some embodiments, the carrier polypeptide and the functional RNA molecule are provided at a molar ratio of about 3:1 to about 8:1 (such as about 4:1). In some embodiments, the cell-targeting segment is configured to bind to a mammalian cell, which may be a diseased cell (such as a cancer cell). In some embodiments, the cell-targeting segment is configured to bind to a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the resulting nanoparticles is about 100 nm or less (such as about 60 nm or less, or about 50 nm or less).

In another aspect, there is provided a method of making a nanoparticle composition comprising combining a double-stranded siRNA molecule with the small-molecule drug to complex the drug to the siRNA molecule; and combining a carrier polypeptide with the siRNA molecule complexed with the small-molecule drug, wherein the carrier polypeptide comprises a cell-targeting segment, a cell-penetrating segment, and an oligonucleotide-binding segment. In some embodiments, the small-molecule drug intercalates the siRNA molecule. In some embodiments, the nanoparticle composition is sterile filtered or lyophilized. In some embodiments, the siRNA molecule is about 10 nucleotides to about 100 nucleotides in length. In some embodiments, the siRNA molecule and the small-molecule drug are provided at a molar ratio of about 1:1 to about 1:60. In some embodiments, the carrier polypeptide and the siRNA molecule are provided at a molar ratio of about 3:1 to about 8:1 (such as about 4:1). In some embodiments, the cell-targeting segment is configured to bind to a mammalian cell, which may be a diseased cell (such as a cancer cell). In some embodiments, the cell-targeting segment is configured to bind to a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the resulting nanoparticles is about 100 nm or less (such as about 60 nm or less, or about 50 nm or less).

In another aspect, there is provided a method of making a nanoparticle composition comprising combining a double-stranded siRNA molecule with the small-molecule chemotherapeutic agent to complex the chemotherapeutic agent to the siRNA molecule; and combining a carrier polypeptide with the siRNA molecule complexed with the small-molecule chemotherapeutic agent, wherein the carrier polypeptide comprises a cell-targeting segment, a cell-penetrating segment, and an oligonucleotide-binding segment. In some embodiments, the chemotherapeutic agent intercalates the siRNA molecule. In some embodiments, the nanoparticle composition is sterile filtered or lyophilized. In some embodiments, the siRNA molecule is about 10 nucleotides to about 100 nucleotides in length. In some embodiments, the siRNA molecule and the small-molecule chemotherapeutic agent are provided at a molar ratio of about 1:1 to about 1:60. In some embodiments, the carrier polypeptide and the siRNA molecule are provided at a molar ratio of about 3:1 to about 8:1 (such as about 4:1). In some embodiments, the cell-targeting segment is configured to bind to a mammalian cell, which may be a diseased cell (such as a cancer cell). In some embodiments, the cell-targeting segment is configured to bind to a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the resulting nanoparticles is about 100 nm or less (such as about 60 nm or less, or about 50 nm or less). In some embodiments, the chemotherapeutic agent is an anthracycline (such as doxorubicin) or an alkylating agent or an alkylating-like agent.

In another aspect, there is provided a method of making a nanoparticle composition comprising combining a double-stranded siRNA molecule with the small-molecule chemotherapeutic agent to complex the chemotherapeutic agent to the siRNA molecule, wherein the siRNA molecule comprises at least one 5′-triphosphate end; and combining a carrier polypeptide with the siRNA molecule complexed with the small-molecule chemotherapeutic agent, wherein the carrier polypeptide comprises a cell-targeting segment, a cell-penetrating segment, and an oligonucleotide-binding segment. In some embodiments, the chemotherapeutic agent intercalates the siRNA molecule. In some embodiments, the nanoparticle composition is sterile filtered or lyophilized. In some embodiments, the siRNA molecule is about 10 nucleotides to about 100 nucleotides in length. In some embodiments, the siRNA molecule and the small-molecule chemotherapeutic agent are provided at a molar ratio of about 1:1 to about 1:60. In some embodiments, the carrier polypeptide and the siRNA molecule are provided at a molar ratio of about 3:1 to about 8:1 (such as about 4:1). In some embodiments, the cell-targeting segment is configured to bind to a mammalian cell, which may be a diseased cell (such as a cancer cell). In some embodiments, the cell-targeting segment is configured to bind to a target molecule on the surface of a cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the resulting nanoparticles is about 100 nm or less (such as about 60 nm or less, or about 50 nm or less). In some embodiments, the chemotherapeutic agent is an anthracycline (such as doxorubicin) or an alkylating agent or an alkylating-like agent.

In another aspect, there is provided a method of making a nanoparticle composition comprising combining a double-stranded siRNA molecule with the small-molecule chemotherapeutic agent to complex the chemotherapeutic agent to the siRNA molecule; and combining HerPBK10 with the siRNA molecule complexed with the small-molecule chemotherapeutic agent. In some embodiments, the chemotherapeutic agent intercalates the siRNA molecule. In some embodiments, the nanoparticle composition is sterile filtered or lyophilized. In some embodiments, the si RNA molecule is about 10 nucleotides to about 100 nucleotides in length. In some embodiments, the siRNA molecule and the small-molecule chemotherapeutic agent are provided at a molar ratio of about 1:1 to about 1:60. In some embodiments, the carrier polypeptide and the siRNA molecule are provided at a molar ratio of about 3:1 to about 8:1 (such as about 4:1). In some embodiments, the average size of the resulting nanoparticles is about 100 nm or less (such as about 60 nm or less, or about 50 nm or less). In some embodiments, the chemotherapeutic agent is an anthracycline (such as doxorubicin) or an alkylating agent or an alkylating-like agent.

### Cancer Treatments

The compositions comprising the therapeutic complex described herein or the nanoparticle compositions described herein can be useful for the treatment of cancer in a subject by administering an effective amount of a composition comprising the nanoparticles to the subject, thereby killing the cancer cells. The cell-targeting segment of the carrier polypeptide can target a molecule on the surface of a cancer cell, thereby delivering a chemotherapeutic agent (e.g., the functional RNA molecule and the small-molecule drug) to the cancer cells. In some embodiments, the cancer is metastatic. In some embodiments, the therapeutic complex or the nanoparticle composition is used in the manufacture of a medicament for the treatment of cancer.

In some embodiments, the cancer is a HER3+ cancer. A Her cell-targeting segment, for example, can bind HER3 present on the surface of the HER3+ cancer cells to target the nanoparticles to the cancer cells. In some embodiments, the cancer is a c-MET+ cancer. An InlB cell-targeting segment, for example, can bind c-MET present on the surface of the c-MET+ cancer cell to target the nanoparticles to the cancer cells.

In some embodiments, an effective amount of a composition comprising the nanoparticles is administered to subject to treat a head and neck cancer, a pancreatic cancer, a breast cancer, an ovarian cancer, a glial cancer, a cervical cancer, a gastric cancer, a skin cancer, a colon cancer, a rectal cancer, a lung cancer, a kidney cancer, a prostate cancer, or a thyroid cancer. Many cancers exhibit upregulated expression for a particular cell surface molecule. One or more of such upregulated molecules are preferred targets for the cell-targeting segment of the carrier protein.

In some embodiments, the method of treating a subject with cancer further comprises a secondary therapy, such as radiation therapy or surgery. Thus, in some embodiments, the composition comprising the nanoparticles described herein is administered to a subject with cancer as a neoadjuvant therapy.

In some embodiments, the subject has not undergone chemotherapy or radiation therapy prior to administration of the nanoparticles described herein. In some embodiments, the subject has undergone chemotherapy or radiation therapy.

In some embodiments, the nanoparticle composition described herein is administered to a subject. In some embodiments, the nanoparticle composition is administered to a subject for in vivo delivery to targeted cells. Generally, dosages and routes of administration of the nanoparticle composition are determined according to the size and condition of the subject, according to standard pharmaceutical practice. In some embodiments, the nanoparticle composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally. In some embodiments, the composition is administered to a subject intravenously.

In some embodiments, the dosage of the nanoparticle composition is a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the composition is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 2, 3, 4, 5, 7, 10, 15, or 20 or more doses).

In some embodiments, an administered dose of the nanoparticle composition is about 200 mg/m², 150 mg/m², 100 mg/m², 80 mg/m², 70 mg/m², 60 mg/m², 50 mg/m², 40 mg/m², 30 mg/m², 20 mg/m², 15 mg/m², 10 mg/m², 5 mg/m², or mg/m² or lower of the small-molecule drug.

In one aspect, there is provided a method of treating a cancer in a subject comprising administering to the subject an effective amount of a composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment. In some embodiments, the cancer is head and neck cancer, a pancreatic cancer, a breast cancer, an ovarian cancer, a glial cancer, a cervical cancer, a gastric cancer, a skin cancer, a colon cancer, a rectal cancer, a lung cancer, a kidney cancer, or a thyroid cancer. In some embodiments, the functional RNA molecule is about 10 nucleotides to about 100 nucleotides in length. In some embodiments, the molar ratio of the functional RNA molecule to the small-molecule drug in the nanoparticle composition is about 1:1 to about 1:60. In some embodiments, the molar ratio of the carrier polypeptide to the functional RNA molecule in the composition is about 3:1 to about 8:1 (such as about 4:1). In some embodiments, the cell-targeting segment binds a cancer cell. In some embodiments, the cell-targeting segment binds a target molecule on the surface of the cancer cell, which may be a receptor (such as HER3 or c-MET). In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less).

In one aspect, there is provided a method of treating a HER3+ cancer in a subject comprising administering to the subject an effective amount of a composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment. In some embodiments, the cancer is head and neck cancer, a pancreatic cancer, a breast cancer, an ovarian cancer, a glial cancer, a cervical cancer, a gastric cancer, a skin cancer, a colon cancer, or a rectal cancer. In some embodiments, the functional RNA molecule is about 10 nucleotides to about 100 nucleotides in length. In some embodiments, the molar ratio of the functional RNA molecule to the small-molecule drug in the nanoparticle composition is about 1:1 to about 1:60. In some embodiments, the molar ratio of the carrier polypeptide to the functional RNA molecule in the composition is about 3:1 to about 8:1 (such as about 4:1). In some embodiments, the cell-targeting segment binds a HER3+ cancer cell. In some embodiments, the cell-targeting segment binds HER3. In some embodiments, the cell-penetrating segment comprises a penton base polypeptide or a variant thereof. In some embodiments, the oligonucleotide-binding segment is positively charged, such as a polylysine. In some embodiments, the carrier polypeptide is HerPBK10. In some embodiments, the average size of the nanoparticles in the composition is about 100 nm or less (such as about 60 nm or less, or about 50 nm or less).

In one aspect, there is provided a method of treating a c-MET+ cancer in a subject comprising administering to the subject an effective amount of a composition comprising nanoparticles comprising a carrier polypeptide and a functional RNA molecule complexed with a small-molecule drug, wherein the carrier polypeptide comprises a cell-targeting segment, a cell-penetrating segment, and an oligonucleotide-binding segment. In some embodiments, the cancer is head and neck cancer, a pancreatic cancer, a breast cancer, an ovarian cancer, a gastric cancer, a colon cancer, a rectal cancer, a lung cancer, a kidney cancer, or a thyroid cancer. In some embodiments, the functional RNA molecule is about 10 nucleotides to about 100 nucleotides in length. In some embodiments, the molar ratio of the functional RNA molecule to the small-molecule drug in the nanoparticle composition is about 1:1 to about 1:60. In some embodiments, the molar ratio of the carrier polypeptide to the functional RNA molecule in the composition is about 3:1 to about 8:1 (such as about 4:1). In some embodiments, the cell-targeting segment binds a c-MET+ cancer cell. In some embodiments, the cell-targeting segment binds c-MET. In some embodiments, the cell-penetrating segment comprises a penton base polypeptide or a variant thereof. In some embodiments, the oligonucleotide-binding segment is positively charged, such as a polylysine. In some embodiments, the average size of the nanoparticles in the composition is about 100 nm or less (such as about 60 nm or less, or about 50 nm or less).

In one aspect, there is provided a method of treating a HER3+ cancer in a subject comprising administering to the subject an effective amount of a composition comprising nanoparticles comprising HerPBK10 and a functional RNA molecule complexed with a small-molecule drug. In some embodiments, the cancer is head and neck cancer, a pancreatic cancer, a breast cancer, an ovarian cancer, a glial cancer, a cervical cancer, a gastric cancer, a skin cancer, a colon cancer, prostate cancer, kidney cancer, or a rectal cancer. In some embodiments, the functional RNA molecule is about 10 nucleotides to about 100 nucleotides in length. In some embodiments, the molar ratio of the functional RNA molecule to the small-molecule drug in the nanoparticle composition is about 1:1 to about 1:60. In some embodiments, the molar ratio of the carrier polypeptide to the functional RNA molecule in the composition is about 3:1 to about 8:1 (such as about 4:1). In some embodiments, the average size of the nanoparticles in the composition is about 100 nm or less (such as about 60 nm or less, or about 50 nm or less).

### Pharmaceutical Compositions

In some embodiments, the compositions described herein are formulated as pharmaceutical compositions comprising a plurality of nanoparticles described herein and a pharmaceutically acceptable excipient.

In some embodiments, the pharmaceutical composition is a solid, such as a powder. The powder can be formed, for example, by lyophilizing the nanoparticles in solution. The powder can be reconstituted, for example by mixing the powder with an aqueous liquid (e.g., water or a buffer). In some embodiments, the pharmaceutical composition is a liquid, for example nanoparticles suspended in an aqueous solution (such as physiological saline or Ringer's solution). In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer.

Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

### Articles of Manufacture and Kits

Also provided are articles of manufacture comprising the compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions (or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### EXEMPLARY EMBODIMENTS

### Embodiment 1

A composition, comprising a functional RNA molecule complexed with a small-molecule drug, wherein the functional RNA molecule modulates expression of a target protein.

### Embodiment 2

A composition, comprising a functional RNA molecule comprising at least one complementary region intercalated with a small-molecule drug.

### Embodiment 3

The composition of embodiment 2, wherein the functional RNA molecule modulates expression of a target protein.

### Embodiment 4

The composition of any one of embodiments 1-3, comprising a liposome containing the functional RNA molecule and the small-molecule drug.

### Embodiment 5

The composition of embodiment 4, wherein the liposome comprises a cell-targeting segment.

### Embodiment 6

A composition comprising nanoparticles comprising a carrier polypeptide and a functional RNA molecule complexed with a small-molecule drug, wherein the carrier polypeptide comprises a cell-penetrating segment and an oligonucleotide-binding segment.

### Embodiment 7

The composition of embodiment 6, wherein the molar ratio of carrier polypeptide to functional RNA molecule in the composition is about 3:1 to about 8:1.

### Embodiment 8

The composition of any one of embodiments 1-7, wherein the small-molecule drug is intercalated into the functional RNA molecule, and wherein the functional RNA molecule comprises at least one complementary region.

### Embodiment 9

The composition of any one of embodiments 6-8, wherein the cell-penetrating segment comprises a penton base polypeptide or a variant thereof.

### Embodiment 10

The composition of any one of embodiments 6-9, wherein the oligonucleotide-binding segment is positively charged.

### Embodiment 11

The composition of any one of embodiments 6-10, wherein the oligonucleotide-binding segment comprises polylysine.

### Embodiment 12

The composition of any one of embodiments 6-10, wherein the oligonucleotide-binding segment comprises decalysine.

### Embodiment 13

The composition of any one of embodiments 6-12, wherein the average size of the nanoparticles in the composition is about 100 nm or less.

### Embodiment 14

The composition of any one of embodiments 6-13, wherein the carrier polypeptide further comprises a cell-targeting segment.

### Embodiment 15

The composition of embodiment 5 or 14, wherein the cell-targeting segment binds a mammalian cell.

### Embodiment 16

The composition of any one of embodiments 5, 14, or 15, wherein the cell-targeting segment binds a diseased cell.

### Embodiment 17

The composition of any one of embodiments 5 and 14-16, wherein the cell-targeting segment binds a cancer cell.

### Embodiment 18

The composition of embodiment 17, wherein the cancer cell is a HER3+ cancer cell or a c-MET+ cancer cell.

### Embodiment 19

The composition of embodiment 17 or 18, wherein the cancer cell is a head and neck cancer cell, a pancreatic cancer cell, a breast cancer cell, a glial cancer cell, an ovarian cancer cell, a cervical cancer cell, a gastric cancer cell, a skin cancer cell, a colon cancer cell, a rectal cancer cell, a lung cancer cell, a kidney cancer cell, a prostate cancer cell, or a thyroid cancer cell.

### Embodiment 20

The composition of any one of embodiments 5 and 14-19, wherein the cell-targeting segment binds a target molecule on the surface of a cell.

### Embodiment 21

The composition of any one of embodiment 5 and 14-20, wherein the cell-targeting segment binds a receptor on the surface of a cell.

### Embodiment 22

The composition of any one of embodiments 5 and 14-21, wherein the cell-targeting segment binds HER3 or c-MET.

### Embodiment 23

The composition of any one of embodiments 5 and 14-22, wherein the cell-targeting segment comprises a ligand that specifically binds to a receptor expressed on the surface of a cell.

### Embodiment 24

The composition of any one of embodiments 5 and 14-23, wherein the cell-targeting segment comprises:
i. a heregulin sequence or a variant thereof; or
ii. an internalin B sequence or a variant thereof.

### Embodiment 25

The composition of any one of embodiments 5 and 14-24, wherein the cell-targeting segment comprises a receptor binding domain of heregulin-α

### Embodiment 26

The composition of any one of embodiments 1-25, wherein at least a portion of the functional RNA molecule is double stranded.

### Embodiment 27

The composition of any one of embodiments 1-25, wherein the functional RNA molecule is single stranded and comprises at least one self-complementary region.

### Embodiment 28

The composition of any one of embodiments 1-27, wherein the functional RNA molecule is siRNA, shRNA, miRNA, circularRNA (circRNA), rRNA, Piwi-interacting RNA (piRNA), toxic small RNA (tsRNA), or a ribozyme.

### Embodiment 29

The composition of any one of embodiments 1-28, wherein the functional RNA molecule is a siRNA molecule or a shRNA molecule.

### Embodiment 30

The composition of any one of embodiments 1-29, wherein the functional RNA molecule has at least one triphosphate 5′-end.

### Embodiment 31

The composition of any one of embodiments 1-30, wherein the functional RNA molecule is about 10 nucleotides to about 100 nucleotides in length.

### Embodiment 32

The composition of any one of embodiments 1-31, wherein the molar ratio of the functional RNA molecule to the small-molecule drug in the composition is about 1:1 to about 1:60.

### Embodiment 33

The composition of any one of embodiments 1-32, wherein the molar ration of functional RNA molecule to the small-molecule drug in the composition is about 1:5 to about 1:60.

### Embodiment 34

The composition of any one of embodiments 1-33, wherein the molar ration of functional RNA molecule to the small-molecule drug in the composition is about 1:10 to about 1:60.

### Embodiment 35

The composition of any one of embodiments 1-34, wherein the small-molecule drug is a chemotherapeutic agent.

### Embodiment 36

The composition of any one of embodiments 1-35, wherein the small-molecule drug is an anthracycline.

### Embodiment 37

The composition of any one of embodiments 1-36, wherein the small-molecule drug is doxorubicin.

### Embodiment 38

The composition of any one of embodiments 1-36 wherein the small-molecule drug is an alkylating agent or an alkylating-like agent.

### Embodiment 39

The composition of any one of embodiments 1-36 and 38, wherein the small-molecule drug is of Carboplatin, Carmustine, Cisplatin, Cyclophosphamide, Melphalan, Procarbazine, or Thiotepa.

### Embodiment 40

The composition of any one of embodiments 1-39, wherein the composition is sterile.

### Embodiment 41

The composition of any one of embodiments 1-40, wherein the composition is a liquid composition.

### Embodiment 42

The composition of any one of embodiments 1-41, wherein the composition is a dry composition.

### Embodiment 43

The composition of embodiment 42, wherein the composition is lyophilized.

### Embodiment 44

A pharmaceutical composition comprising the composition of any one of embodiments 1-43, further comprising a pharmaceutically acceptable excipient.

### Embodiment 45

An article of manufacture comprising the composition of any one of embodiments 1-44 in a vial.

### Embodiment 46

The article of manufacture of embodiment 45, wherein the vial is sealed.

### Embodiment 47

A kit comprising the composition of any one of embodiments 1-44, and an instruction for use.

### Embodiment 48

A method of treating a cancer in a subject comprising administering an effective amount of the composition according to any one of embodiments 1-44 to the subject.

### Embodiment 49

The method of embodiment 48, wherein the cancer is a HER3+ cancer or a c-MET+ cancer.

### Embodiment 50

The method of embodiment 48 or 49, wherein the cancer is a head and neck cancer, a pancreatic cancer, a breast cancer, an ovarian cancer, a glial cancer, a cervical cancer, a gastric cancer, a skin cancer, a colon cancer, a rectal cancer, a lung cancer, a kidney cancer, a prostate cancer, or a thyroid cancer.

### Embodiment 51

A method of making a composition, comprising combining a small-molecule drug with a functional RNA molecule, wherein the small-molecule drug intercalates into the functional RNA molecule.

### Embodiment 52

A method of making a nanoparticle composition comprising combining a carrier polypeptide, a functional RNA molecule, and a small-molecule drug, wherein the carrier polypeptide comprises a cell-penetrating segment and an oligonucleotide-binding segment.

### Embodiment 53

The method of embodiment 52, comprising:
combining the functional RNA molecule with the small-molecule drug to complex the small-molecule drug to the functional RNA molecule, and
combining the carrier polypeptide with the functional RNA molecule complexed with the small-molecule drug.

### Embodiment 54

The method of embodiment 52 or 53, wherein the small-molecule drug intercalates the functional RNA molecule.

### Embodiment 55

The method of any one of embodiments 51-54, comprising removing unbound small-molecule drug.

### Embodiment 56

The method of any one of embodiments 51-55, further comprising sterile filtering the nanoparticle composition.

### Embodiment 57

The method of any one of embodiments 51-56, further comprising lyophilizing the nanoparticle composition.

### Embodiment 58

A method of simultaneously modulating expression of a target protein and inhibiting growth of a cell, comprising administering an effective amount of the composition according to any one of embodiments 1-44 to the cell.

### Embodiment 59

A method of killing a cell, comprising administering an effective amount of the composition according to any one of embodiments 1-44 to the cell.

### Embodiment 60

A method of simultaneously stimulating an immune response and killing a cell, comprising administering an effective amount of the composition according to any one of embodiments 1-44 to the cell, wherein the functional RNA molecule modulates expression of an immune checkpoint protein.

### EXAMPLES

The examples provided herein are included for illustrative purposes only and are not intended to limit the scope of the invention.

### Example 1: Nanoparticle Assembly

Nanoparticles comprising a carrier polypeptide, a functional RNA molecule, and a small-molecule drug (such as doxorubicin) can be assembled using the following methods.

Single stranded siRNA and its complement RNA molecule can be annealed by incubating equal molar ratios of each oligonucleotide in boiling water for 5 minutes. The oligonucleotides can then be cooled at room temperature for 30 minutes.

The double-stranded, annealed siRNA molecules can then be incubated with doxorubicin HCl at a molar ratio of 1:40 RNA:Dox at room temperature for 30 minutes.

The doxorubicin-bound siRNA molecules can then be incubated with a carrier polypeptide (such as HerPBK10) comprising a Her cell-targeting segment, a PB cell-penetrating segment, and a decalysine (“K10”) oligonucleotide binding segment at a molar ratio of 4:1 HerPBK10:siRNA-doxorubicin (thus a molar ratio of 4:1:40 HerPBK10:siRNA:doxorubicin) in HEPES Buffered Saline (HBS). The mixture of carrier polypeptide and doxorubicin-bound siRNA can be rocked for 2 hours on ice, thereby forming the nanoparticles.

The resulting nanoparticles can be subjected to ultracentrifugation. Specifically, 12 mL of sterile HBS can be added to a 50 kD cut-off Centrifugal Filter (Amicon Ultra-15) that may have been pre-incubated in sterile, 10% glycerol for 24 hours. The nanoparticle mixtures can be added to the cold HBS in the centrifugal filer. The filter tubes can be spun for 10-20 minutes at 2500 RPM (5000×g) in a Beckman J6-HC centrifuge until the final volume was between 200 μL and 500 μL. The concentrated nanoparticles can then be transferred to a 1.7 mL microfuge tube.

Nanoparticles without the nanoparticle drug can be prepared by incubating HerPBK10 with siRNA that is not complexed to the small-molecule drug (see, for example US. Patent Application No. 2012/0004181). Other comparative nanoparticles can be formed, for example by incubating HerPBK10 with double-stranded DNA that is complexed to the small-molecule drug (see, for example, U.S. Pat. No. 9,078,927).

### Example 2: Use of Nanoparticles to Kill Cancer Cells and Chemotherapeutic Drug Resistant Cancer Cells

Nanoparticles with doxorubicin-bound siRNA, nanoparticles with siRNA and no doxorubicin, or nanoparticles with doxorubicin-bounds dsDNA can be compared for their ability to kill various types of cancer cells.

Various doses of nanoparticles can be incubated with either MDA-MB-435 (human cancer) cells, BT474 (human breast cancer) cells, U251 (human glioma) cells, SKOV3 (human ovarian cancer) cells, LNCaP-GFP (human prostate cancer) cells, or RANKL (human bone-metastatic prostate cancer cells).

Relative cell survival after exposure to the described compositions can be measured using a cell viability assay. The cells can be plated in black-walled, clear-bottom, 96-well plates. 48 hours later, the media can be aspirated and replaced with complete media and the indicated concentrations of nanoparticles at a total volume of 40 μL. Plates can be rocked for 4 hours at 37° C. and 5% CO₂ and then 60 μL of complete media can be added to each well to bring the total volume to 100 μL and the incubation was continued, without rocking, for 44 hours at 37° C. and 5% CO₂. At the conclusion of the incubation, relative cell viability can be determined via MTS assay (Promega) according to manufacturer's instructions. Specifically, the media can be removed from the wells and 100 μL of fresh complete media can be added to each well. 20 μl of the prepared MTS reagent can be added to each well. The plate can then be incubated with rocking at 37° C. and 5% CO₂ and readings were taken of the plate at 1, 2, and 3 hours at 490 nm on spectrophotometer. The results can be shown in terms of the following ratio: number of cells that survived in the treatment group divided by the number of cells that survived in the untreated group. Thus, cell survival of 1.0 indicates that the treated cells and the untreated cells survived to the same extent, whereas a ratio of 0.2 means that as compared with the untreated cell group, only 20% of the treated cells survived.

### Example 3: Assembly of a Therapeutic Complex with Doxorubicin Intercalated into Double Stranded siRNA

Two different therapeutic complexes were formed by combining doxorubicin with double stranded siRNA (“siRNA1,” 21 bases in length; and “siRNA2,” 21 bases in length). For the first therapeutic complex, 10 μL of doxorubicin-HCl (Sigma-Aldrich; 10 mM stock solution) and 5.2 μL of siRNA1 (0.48 mM stock solution) were combined with 465 μL of HEPES buffered saline (HBS). For the second therapeutic complex, 10 μL of doxorubicin (10 mM stock solution) and 25 μL of siRNA1 (0.1 mM stock solution) were combined with 484.8 μL of HBS. Each therapeutic complex sample was incubated for 30 minutes at room temperature while rocking before being centrifuged using a 10K MWCO filter to remove unbound doxorubicin. As a control, 10 μL of doxorubicin (10 mM stock solution) was added to 490 μL of HBS, but was not passed through the filter. Samples (10 μL) of the therapeutic complex before filtration, the rententate, and the filtrate were analyzed on a 1% agarose gel, as shown in FIG. 2. Lanes and corresponding samples are indicated in Table 1:

TABLE 1
Lane Samples for FIG. 2

| Lane | Sample |
|---|---|
| 1 | Ladder |
| 2 | Dox:siRNA2 pre-filtration |
| 3 | Dox:siRNA1 pre-filtration |
| 4 | Empty |
| 5 | Dox:siRNA2 retentate |
| 6 | Dox:siRNA1 retentate |
| 7 | Dox:siRNA2 filtrate |
| 8 | Dox:siRNA1 filtrate |

As shown in FIG. 2, siRNA is detected in lanes 2, 3, 5, and 6, but not in lanes 7 and 8. This indicates that the siRNA for both complexes (Dox:siRNA1 and Dox:siRNA2) were retained in the retentate, and did not pass through the filter into the filtrate.

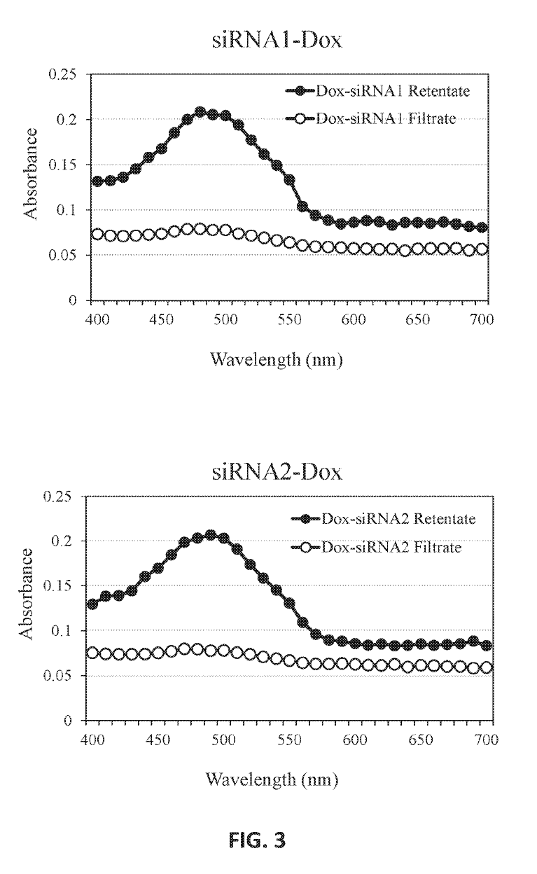

Absorbance from 400 nm to 700 nm was also measured for the retantate (100 μL) and filtrate (100 μL) of each sample. These results are shown in FIG. 3 (closed circles indicate the retentate and open circles indicate the filtrate). For both the Dox:siRNA1 complex and the Dox:siRNA2 complex, the retentate had a maximum absorbance of about 0.21 at about 480 nm, the absorbance maximum for doxorubicin. In contrast, the filtrate of the Dox:siRNA1 and Dox:siRNA2 complexes did not have a significant peak at about 490 nm. This indicates that the doxorubicin in the samples was retained in the retentate, and was therefore complexed to the siRNA.

### Example 4: Gene Silencing and Decreased Cell Viability Using a Therapeutic Complex

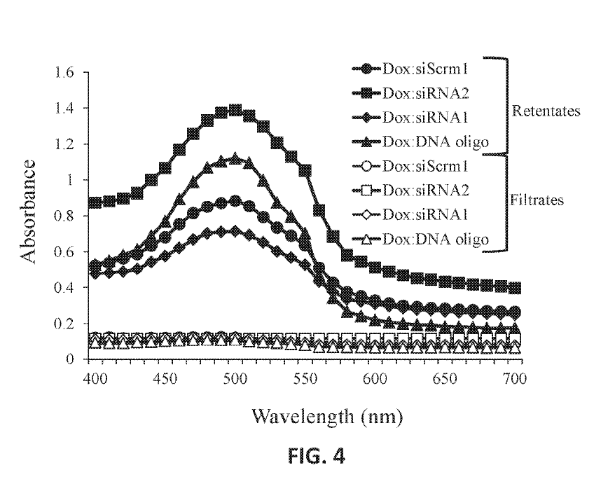

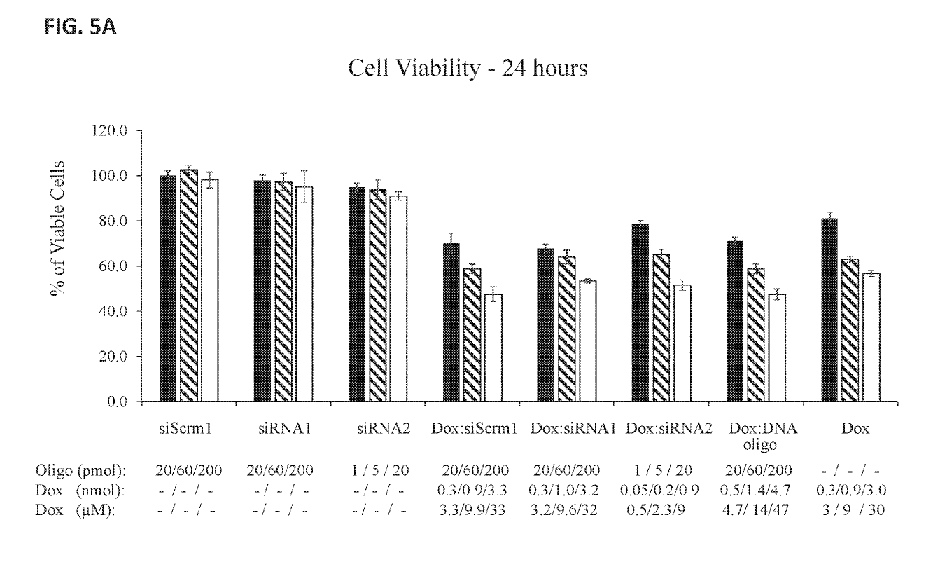

Complexes including a scrambled non-functional, double-stranded RNA molecule (“siScrm1,” 21 bases in length), functional double stranded siRNA (“siRNA1,” 21 bases in length; or “siRNA2,” 21 bases in length), or double-stranded DNA (“DNA oligo,” 30 bases in length) complexed with doxorubicin were formed by combining 100 nmol of doxorubicin with 2.5 nmol of RNA or DNA. For the first complex, 10 μL of doxorubicin-HCl (Sigma-Aldrich; 10 mM stock solution) and 25 μL siScrm1 (0.2 mM stock solution) were combined with 365 μL HBS. For the second complex, 10 μL of doxorubicin (20 mM stock solution) and 5.2 μL siRNA1 (0.48 mM stock solution) were combined with 384.8 μL HBS. For the third complex, 10 μL of doxorubicin (20 mM stock solution) and 25 μL siRNA2 (0.1 mM stock solution) were combined with 365 μL HBS. For the fourth complex, 10 μL of doxorubicin (20 mM stock solution) and 2.5 μL DNA oligo (1 mM stock solution) were combined with 387.5 μL HBS. Each sample was incubated for 30 minutes at room temperature while rocking before being centrifuged using a 10K MWCO filter to remove unbound doxorubicin. Absorbance from 400 nm to 700 nm was also measured for the retantate (100 μL) and filtrate (100 μL) of each sample. These results are shown in FIG. 4 (closed symbols indicate the retentate and open symbols indicate the filtrate). Each retentate sample had an absorbance peak at about 480 nm (Dox:siScrm1 maximum absorbance ˜0.9; Dox:siRNA1 maximum absorbance ˜0.7; Dox:siRNA2 maximum absorbance ˜1.4; Dox:DNA oligo maximum absorbance ˜1.1). The filtrate of each sample did not have a significant peak indicating the absence of substantial amounts of doxorubicin. Doxorubicin detected in the retentate complexed to the DNA or RNA. Yield for the doxorubicin and the DNA or RNA was calculated, as shown in Table 2. Yield of doxorubicin was determined based on absorbance at 480 nm using a doxorubicin standard curve. Yield of nucleic acid (RNA or DNA) was determined based on absorbance at 260 nm after heating the samples to 85° C.

TABLE 2
Yield of doxorubicin and DNA/RNA in complex

| | | | Dox/Nucleic Acid |
|---|---|---|---|
| | Dox Yield | Nucleic Acid Yield | Ratio |
| Dox:DNA oligo | 70 nmol | 3 nmol | 23.3 |
| Dox:siScrm1 | 50 nmol | 3 nmol | 16.7 |
| Dos:siRNA1 | 40 nmol | 3 nmol | 16.0 |
| Dox:siRNA2 | 90 nmol | 2 nmol | 45 |

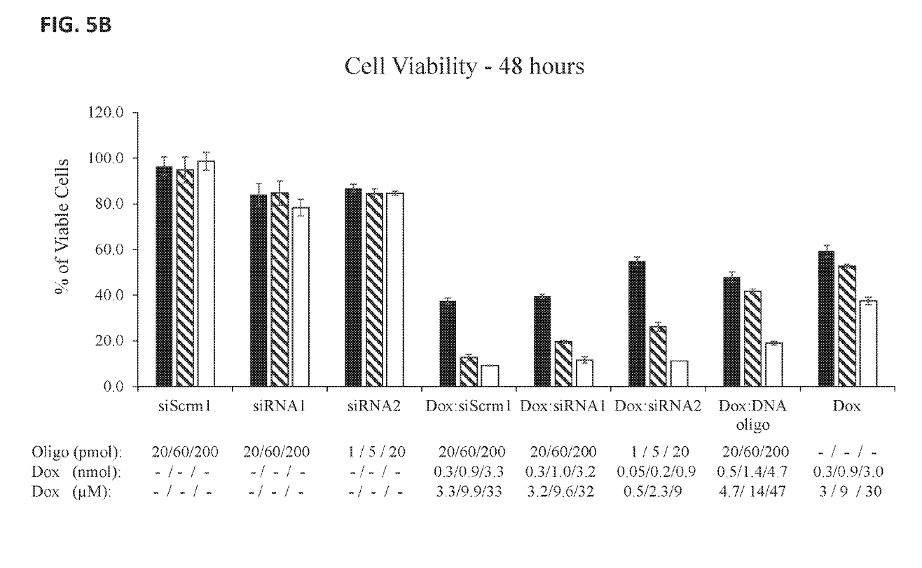

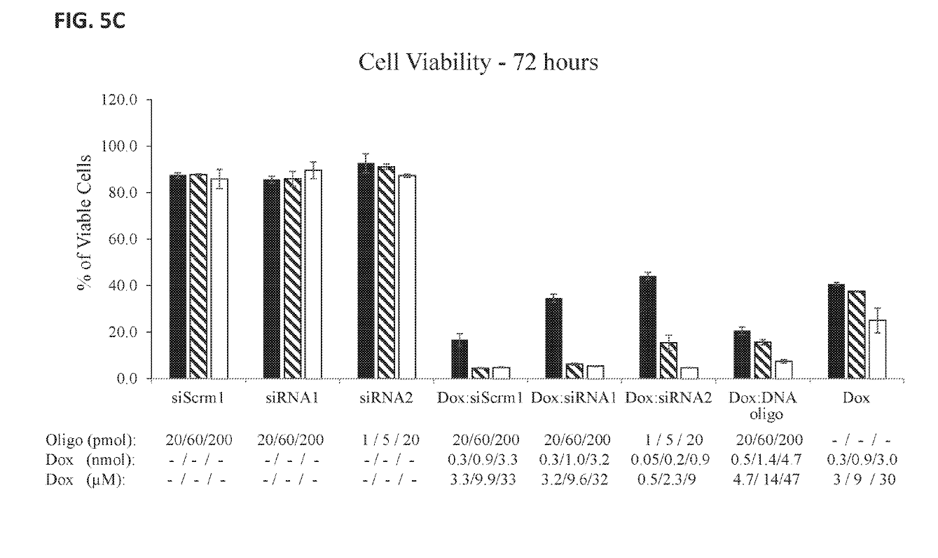

To measure the effect of the complexes on cell viability, the formed complexes were transfected into JIMT1 cells (trastuzumb-resistant human breast cancer). Approximately 10,000 cells per well were plated in 96-well plates, maintained in RPMI 1640 medium with 100% fetal bovine serum, 100 U/mL penicillin, 100 μg/mL streptomycin at 37° C. under 5% CO₂. After 24 hours, the culture media was replaced with Opti-MEM I reduced serum medium (Invitrogen Life Technologies). RNAiMax lipofectamine (Invitrogen Life Technologies) was used as a carrier for siRNA, Dox:siRNA complexes, and the Dox:DNA oligo complex delivery. Doxorubicin was administered to the control sample without the lipofectamine. Three hours following transfection, the medium in each sample was replaced with complete culture media. After 24, 48, or 72 hours, relative cell viability was determined by quantifying ATP using Celltiter Glo Luminescent Cell Viability Kit (Promega), according to the manufacturer's instructions. Experiments were conducted in triplicate. Results are shown in FIG. 5A (24 hours), 5B (48 hours), and SC (72 hours).

The RNA alone (either siScrm1, siRNA1, or siRNA2) had little or no effect on cell viability. There is a small decrease in cell viability after 72 hours, but this is not dose dependent and is attributable to natural cell death during the course of the experiment. The double stranded RNA complexed with doxorubicin, or doxorubicin alone, showed a dose-dependent decrease in cell viability after 24, 48, and 72 hours. Surprisingly, the therapeutic complex containing siRNA and doxorubicin resulted in a significant decrease in cell viability compared to doxorubicin alone (particularly visible at the 48 and 72 hour time points). This is further surprising considering that the doxorubicin dosage of the Dox:siRNA2 complex administered to the cells was substantially lower than the dosage of doxorubicin alone (0.05/0.2/0.9 nmol compared to 0.3/0.9/3.0 nmol). Further, the Dox:siRNA complexes resulted in a decrease in cell viability at least as much as the Dox:DNA oligo complex, even though a lower dosage of doxorubicin was administered.

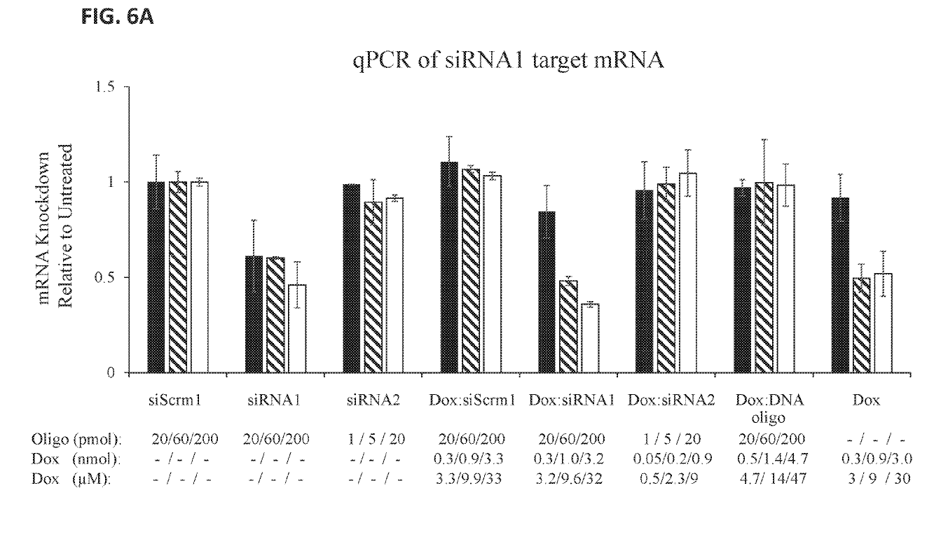

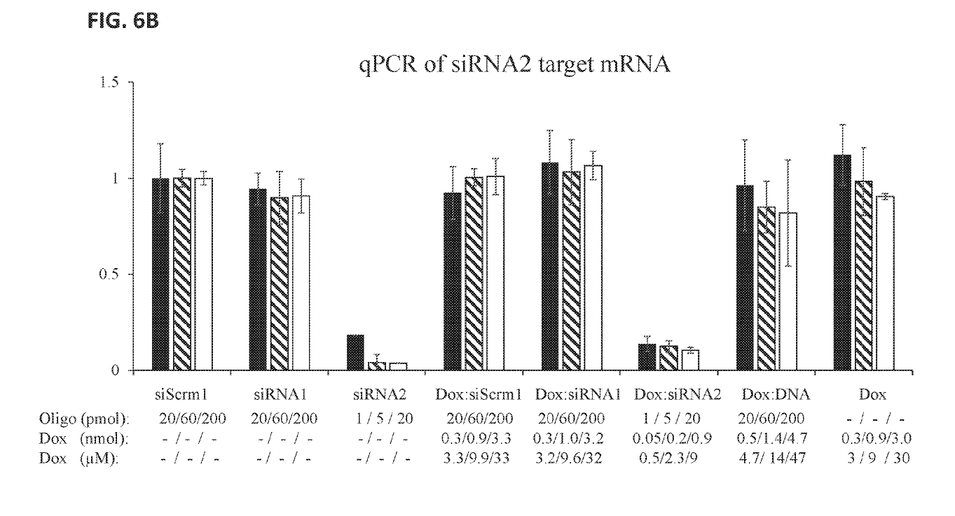

To ensure that the siRNA complexed with doxorubicin remained functional after transfection, RNA targets of the siRNA molecule was quantified using qPCR. Total RNAs were extracted from the transfected JIMT1 cells 24 hours after transfection using TriZol reagent (Invitrogen Life Technologies). Reverse transcription was performed on 1 μg of total RNA using iScript™ cDNA Synthesis Kit (Bio-Rad) according to the manufacturer's instructions. Sets of specific primers (Bio-Rad) and SYBR Green were used for amplification. The qPCR reaction was performed on a Bio-Rad CFX Connect™ instrument (Bio-Rad) as follows: 95° C. for 30 seconds, and then 40 cycles of 95° C. for 10 seconds and 60° C. for 30 seconds. The specificity of the reaction was verified by melt curve analysis Samples were normalized to HPRT1 using the AACt method. Results are shown in FIG. 6A (siRNA1) and 6B (siRNA2).

As expected the siScrm1 and siRNA2 do not decrease siRNA1 target mRNA levels, whereas the siRNA1 does decrease mRNA levels. Further, the dox:siScrm1, dox:siRNA2, and dox:DNA oligo complexes do not impact mRNA levels of the siRNA1 target. In contrast, the dox:siRNA1 complex does cause a significant decrease in siRNA1 target mRNA levels, which indicates that the siRNA1 in the complex remains functional even though the siRNA molecule was complexed with doxorubicin. FIG. 6B shows similar results for the siRNA2 target mRNA, where only the siRNA2 molecule alone and the dox:siRNA2 complex results in more complete silencing of the siRNA2 target mRNA.

These combined results indicate that the dox:siRNA complex is formed, and that the siRNA and doxorubicin remain functional upon administration to a cell.

### Example 5: Gene Silencing and Decreased Cell Viability Using a Therapeutic Complex

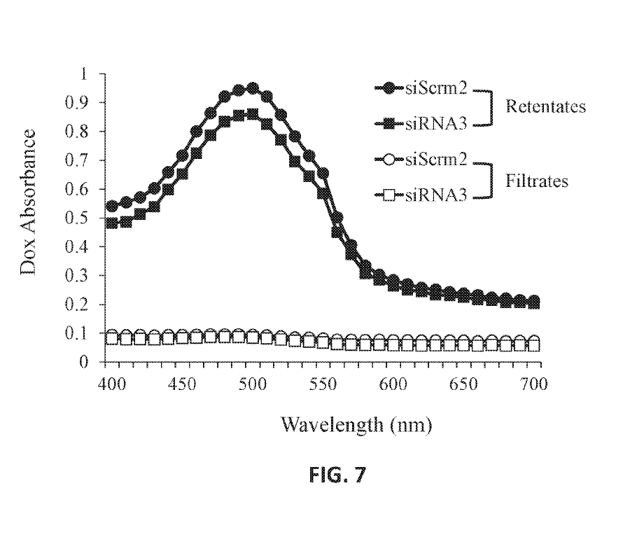

Complexes including a scrambled non-functional, double-stranded RNA molecule (“siScrm2,” 21 bases in length) or functional double stranded siRNA (“siRNA3,” 21 bases in length) complexed with doxorubicin were formed by combining 100 nmol of doxorubicin with 2.5 nmol of RNA. For the first complex, 20 μL of doxorubicin-HCl (Sigma-Aldrich; 5 mM stock solution) and 50 μL siScrm2 (0.05 mM stock solution) were combined with 350 μL HBS. For the second complex, 20 μL of doxorubicin (5 mM stock solution) and 50 μL siRNA3 (0.05 mM stock solution) were combined with 350 μL HBS. Each sample was incubated for 30 minutes at room temperature while rocking before being centrifuged using a 10K MWCO filter to remove unbound doxorubicin. Absorbance from 400 nm to 700 nm was also measured for the retantate (100 μL) and filtrate (100 μL) of each sample. These results are shown in FIG. 7 (closed symbols indicate the retentate and open symbols indicate the filtrate). Each retentate sample had an absorbance peak at about 480 nm (Dox:siScrm2 maximum absorbance ˜0.95; Dox:siRNA3 maximum absorbance ˜0.85). The filtrate of each sample did not have a significant peak, indicating the absence of substantial amounts of doxorubicin. Doxorubicin detected in the retentate was complexed to the DNA or RNA. Yield for the doxorubicin and the RNA was calculated, as shown in Table 3. Yield of doxorubicin was determined based on absorbance at 480 nm using a doxorubicin standard curve. Yield of RNA was determined based on absorbance at 260 nm after heating the samples to 85° C.

TABLE 3
Yield of doxorubicin and RNA in complex

| | | | Dox/RNA |
|---|---|---|---|
| | Dox Yield | Nucleic Acid Yield | Ratio |
| Dox:siScrm2 | 72 nmol | 1.8 nmol | 40:1 |
| Dos:siRNA3 | 63.6 nmol | 2 nmol | 31.8:1 |

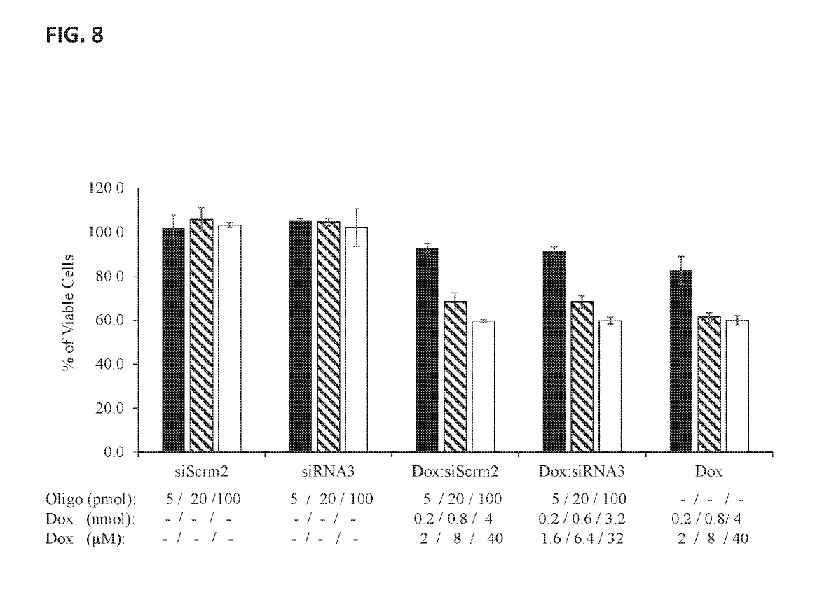

To measure the effect of the complexes on cell viability, the formed complexes were transfected into 4T1-Fluc-Neo/eGFP-Puro cells (mouse mammary carcinoma cells stably expressing FLuc and eGFP). Approximately 10,000 cells per well were plated in 96-well plates, maintained in RPMI 1640 medium with 100, fetal bovine serum, 100 U/mL penicillin, 100 μg/mL streptomycin at 37° C. under 5% CO₂. After 24 hours, the culture media was replaced with Opti-MEM I reduced serum medium (Invitrogen Life Technologies). RNAiMax lipofectamine (Invitrogen Life Technologies) was used as a carrier for siScrm2, dox:siScrm2, siRNA3, or dox:siRNA3 delivery. Doxorubicin was administered to the control sample without the lipofectamine. Three hours following transfection, the medium in each sample was replaced with complete culture media. After 24 hours, relative cell viability was determined by quantifying ATP using Celltiter Glo Luminescent Cell Viability Kit (Promega), according to the manufacturer's instructions. Experiments were conducted in triplicate. Results are shown in FIG. 8.

The RNA alone (either siScrm2 or siRNA3) had little or no effect on cell viability. The double stranded RNA complexed with doxorubicin, or doxorubicin alone, showed a dose-dependent decrease in cell viability after 24 hours.

What is claimed is:

1. A composition, comprising a functional RNA molecule complexed with a small-molecule drug, wherein the functional RNA molecule modulates expression of a target protein.

2. A composition, comprising a functional RNA molecule comprising at least one complementary region intercalated with a small-molecule drug.

3. The composition of claim 2, wherein the functional RNA molecule modulates expression of a target protein.

4. The composition of any one of claims 1-3, comprising a liposome containing the functional RNA molecule and the small-molecule drug.

5. The composition of claim 4, wherein the liposome comprises a cell-targeting segment.

6. A composition comprising nanoparticles comprising a carrier polypeptide and a functional RNA molecule complexed with a small-molecule drug, wherein the carrier polypeptide comprises a cell-penetrating segment and an oligonucleotide-binding segment.

7. The composition of claim 6, wherein the molar ratio of carrier polypeptide to functional RNA molecule in the composition is about 3:1 to about 8:1.

8. The composition of any one of claims 1-7, wherein the small-molecule drug is intercalated into the functional RNA molecule, and wherein the functional RNA molecule comprises at least one complementary region.

9. The composition of any one of claims 6-8, wherein the cell-penetrating segment comprises a penton base polypeptide or a variant thereof.

10. The composition of any one of claims 6-9, wherein the oligonucleotide-binding segment is positively charged.

11. The composition of any one of claims 6-10, wherein the oligonucleotide-binding segment comprises polylysine.

12. The composition of any one of claims 6-10, wherein the oligonucleotide-binding segment comprises decalysine.

13. The composition of any one of claims 6-12, wherein the average size of the nanoparticles in the composition is about 100 nm or less.

14. The composition of any one of claims 6-13, wherein the carrier polypeptide further comprises a cell-targeting segment.

15. The composition of claim 5 or 14, wherein the cell-targeting segment binds a cancer cell.

16. The composition of any one of claim 5, 14, and 15, wherein the cell-targeting segment binds a receptor on the surface of a cell.

17. The composition of any one of claims 5 and 14-16, wherein the cell-targeting segment binds HER3 or c-MET.

18. The composition of any one of claims 5 and 14-17, wherein the cell-targeting segment comprises:
i. a heregulin sequence or a variant thereof; or
ii. an internalin B sequence or a variant thereof.

19. The composition of any one of claims 5 and 14-18, wherein the cell-targeting segment comprises a receptor binding domain of heregulin-α

20. The composition of any one of claims 1-19, wherein at least a portion of the functional RNA molecule is double stranded.

21. The composition of any one of claims 1-20, wherein the functional RNA molecule is single stranded and comprises at least one self-complementary region.

22. The composition of any one of claims 1-21, wherein the functional RNA molecule is a siRNA molecule or a shRNA molecule.

23. The composition of any one of claims 1-22, wherein the functional RNA molecule is about 10 nucleotides to about 100 nucleotides in length.

24. The composition of any one of claims 1-23, wherein the functional RNA molecule decreases expression of an immune checkpoint protein.

25. The composition of any one of claims 1-24, wherein the molar ratio of the functional RNA molecule to the small-molecule drug in the composition is about 1:1 to about 1:60.

26. The composition of any one of claims 1-25, wherein the molar ratio of the functional RNA molecule to the small-molecule drug in the composition is about 1:5 to about 1:60.

27. The composition of any one of claims 1-26, wherein the small-molecule drug is a chemotherapeutic agent.

28. The composition of any one of claims 1-27, wherein the small-molecule drug is an anthracycline, an alkylating agent, or an alkylating-like agent.

29. The composition of any one of claims 1-28, wherein the small-molecule drug is doxorubicin.

30. The composition of any one of claims 1-29, wherein the composition is sterile.

31. The composition of claim 30, wherein the composition is lyophilized.

32. A pharmaceutical composition comprising the composition of any one of claims 1-31, further comprising a pharmaceutically acceptable excipient.

33. An article of manufacture comprising the composition of any one of claims 1-32 in a vial.

34. A kit comprising the composition of any one of claims 1-32 or the article of manufacture of claim 33, and an instruction for use.

35. A method of treating a cancer in a subject comprising administering an effective amount of the composition according to any one of claims 1-32 to the subject.

36. A method of simultaneously modulating expression of a target protein and inhibiting growth of a cell, comprising administering an effective amount of the composition according to any one of claims 1-32 to the cell.

37. A method of simultaneously stimulating an immune response and killing a cancer cell in a subject with cancer, comprising administering an effective amount of the composition according to any one of claims 1-32 to the subject.

38. A method of making a composition, comprising combining a small-molecule drug with a functional RNA molecule, wherein the small-molecule drug intercalates into the functional RNA molecule.

39. A method of making a nanoparticle composition comprising combining a carrier polypeptide, a functional RNA molecule, and a small-molecule drug, wherein the carrier polypeptide comprises a cell-penetrating segment and an oligonucleotide-binding segment.

40. The method of claim 39, comprising:
combining the functional RNA molecule with the small-molecule drug to complex the small-molecule drug to the functional RNA molecule; and
combining the carrier polypeptide with the functional RNA molecule complexed with the small-molecule drug.

41. The method of claim 39 or 40, wherein the small-molecule drug intercalates the functional RNA molecule.

42. The method of any one of claims 39-40, comprising removing unbound small-molecule drug.

43. The method of any one of claims 39-42, further comprising lyophilizing the nanoparticle composition.
